(19) United States
(12) Patent Application Publication
(10) Pub. No.: US 2006/0241100 A1
(43) Pub. Date: Oct. 26, 2006

(54) Acylaminobicyclic heteroaromatic compounds and uses thereof

(75) Inventors: Robert L. Dow, Groton, CT (US); Philip A. Carpino, Groton, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/409,458

(22) Filed: Apr. 20, 2006

Related U.S. Application Data
(60) Provisional application No. 60/673,546, filed on Apr. 20, 2005.

(51) Int. Cl. A61K 31/55; A61K 31/4162; C07D 491/02; C07D 487/02

(52) U.S. Cl. 514/215; 540/578; 548/360.5; 514/406

(57) ABSTRACT

Compounds of Formula (I) are described herein. 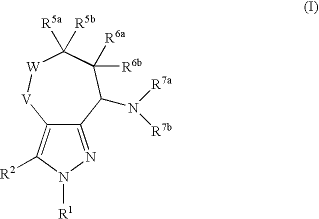 The compounds have been shown to act as cannabinoid receptor ligands and are therefore useful in the treatment of diseases linked to the mediation of the cannabinoid receptors in animals.

This application claims the benefit of U.S. Provisional Application No. 60/673,546 filed on Apr. 20, 2005 and incorporated herein by reference in its entirety.

### FIELD OF THE INVENTION

The present invention relates to acylaminobicyclic compounds. The invention also relates to the use of such compounds as cannabinoid receptor ligands, in particular CB1 receptor antagonists, and uses thereof for treating diseases, conditions and/or disorders modulated by cannabinoid receptor antagonists.

### BACKGROUND

Obesity is a major public health concern because of its increasing prevalence and associated health risks. Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m². See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083 (1998).

The increase in obesity is of concern because of the excessive health risks associated with obesity, including coronary heart disease, strokes, hypertension, type 2 diabetes mellitus, dyslipidemia, sleep apnea, osteoarthritis, gall bladder disease, depression, and certain forms of cancer (e.g., endometrial, breast, prostate, and colon). The negative health consequences of obesity make it the second leading cause of preventable death in the United States and impart a significant economic and psychosocial effect on society. See, McGinnis M, Foege W H., “Actual Causes of Death in the United States,” JAMA, 270, 2207-12 (1993).

Obesity is now recognized as a chronic disease that requires treatment to reduce its associated health risks. Although weight loss is an important treatment outcome, one of the main goals of obesity management is to improve cardiovascular and metabolic values to reduce obesity-related morbidity and mortality. It has been shown that 5-10% loss of body weight can substantially improve metabolic values, such as blood glucose, blood pressure, and lipid concentrations. Hence, it is believed that a 5-10% intentional reduction in body weight may reduce morbidity and mortality.

Currently available prescription drugs for managing obesity generally reduce weight by inducing satiety or decreasing dietary fat absorption. Satiety is achieved by increasing synaptic levels of norepinephrine, serotonin, or both. For example, stimulation of serotonin receptor subtypes 1B, 1D, and 2C and 1- and 2-adrenergic receptors decreases food intake by regulating satiety. See, Bray G A, “The New Era of Drug Treatment. Pharmacologic Treatment of Obesity: Symposium Overview,” Obes Res., 3(suppl 4), 415s-7s (1995). Adrenergic agents (e.g., diethylpropion, benzphetamine, phendimetrazine, mazindol, and phentermine) act by modulating central norepinephrine and dopamine receptors through the promotion of catecholamine release. Older adrenergic weight-loss drugs (e.g., amphetamine, methamphetamine, and phenmetrazine), which strongly engage in dopamine pathways, are no longer recommended because of the risk of their abuse. Fenfluramine and dexfenfluramine, both serotonergic agents used to regulate appetite, are no longer available for use.

More recently, CB1 cannabinoid receptor antagonists/inverse agonists have been suggested as potential appetite suppressants. See, e.g., Arnone, M., et al., “Selective Inhibition of Sucrose and Ethanol Intake by SR141716, an Antagonist of Central Cannabinoid (CB1) Receptors,” Psychopharmacol, 132, 104-106 (1997); Colombo, G., et al., “Appetite Suppression and Weight Loss after the Cannabinoid Antagonist SR141716,” Life Sci., 63, PL113-PL117 (1998); Simiand, J., et al., “SR141716, a CB1 Cannabinoid Receptor Antagonist, Selectively Reduces Sweet Food Intake in Marmose,” Behav. Pharmacol., 9, 179-181 (1998); and Chaperon, F., et al., “Involvement of Central Cannabinoid (CB1) Receptors in the Establishment of Place Conditioning in Rats,” Psychopharmacology, 135, 324-332 (1998). For a review of cannabinoid CB1 and CB2 receptor modulators, see Pertwee, R. G., “Cannabinoid Receptor Ligands: Clinical and Neuropharmacological Considerations, Relevant to Future Drug Discovery and Development,” Exp. Opin. Invest. Drugs, 9(7), 1553-1571 (2000).

Although investigations are on-going, there still exists a need for a more effective and safe therapeutic treatment for reducing or preventing weight-gain.

In addition to obesity, there also exists an unmet need for treatment of alcohol abuse. Alcoholism affects approximately 10.9 million men and 4.4 million women in the United States. Approximately 100,000 deaths per year have been attributed to alcohol abuse or dependence. Health risks associated with alcoholism include impaired motor control and decision making, cancer, liver disease, birth defects, heart disease, drug/drug interactions, pancreatitis and interpersonal problems. Studies have suggested that endogenous cannabinoid tone plays a critical role in the control of ethanol intake. The endogenous CB1 receptor antagonist SR-141716A has been shown to block voluntary ethanol intake in rats and mice. See, Amone, M., et al., “Selective Inhibition of Sucrose and Ethanol Intake by SR141716, an Antagonist of Central Cannabinoid (CB1) Receptors,” Psychopharmacol, 132, 104-106 (1997). For a review, see Hungund, B. L and B. S. Basavarajappa, “Are Anandamide and Cannabinoid Receptors involved in Ethanol Tolerance? A Review of the Evidence,” Alcohol & Alcoholism. 35(2) 126-133, 2000.

Current treatments for alcohol abuse or dependence generally suffer from non-compliance or potential hepatotoxicity; therefore, there is a high unmet need for more effective treatment of alcohol abuse/dependence.

### SUMMARY

The present invention provides compounds of Formula (I): 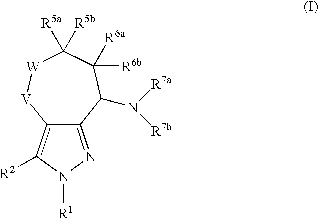
wherein

R¹ and R² are each independently an aryl optionally substituted with one or more substituents, or a heteroaryl optionally substituted with one or more substituents;

V is O and W is CR^{3a}R^{3b}, or V is CR^{3a}R^{3b} and W is N—R⁴;

R^{3a}, R^{3b}, R^{5a}, R^{5b}, R^{6a}, R^{6b}, and R^{7a} are each independently hydrogen, (C₁-C₄)alkyl, or halo-substituted (C₁-C₄)alkyl;

R⁴ is hydrogen, (C₁-C₄)alkyl, halo-substituted (C₁-C₄)alkyl, ((C₁-C₄)alkoxy)-C(O)—, aryl, ((C₁-C₄)alkyl)C(O)—, (aryl)-C(O)—, ((C₁-C₄)alkyl)-SO₂—, or (aryl)-SO₂— (each of the aryl moieties are preferably phenyl);

R^{7b} is

(i) hydrogen,

(ii) (C₁-C₆)alkyl,

(iii) (C₂-C₆)alkenyl,

(iv) halo-substituted (C₁-C₄)alkyl,

(v) —C(O)—(CH₂)ₚR⁸, where p is 0 or 1, and R⁸ is a chemical moiety selected from the group consisting of C₁-C₆)alkyl, (C₂-C₆)alkenyl, (C₁-C₄)alkoxy, (C₃-C₇)cycloalkyl, (C₁-C₄)alkyl)-SO₂—, 3- to 6-membered heterocycle containing one to three heteroatoms independently selected from O, N and S, 5- to 6-membered lactam or lactone, and 5- to 6-membered heteroaryl containing one to three heteroatoms independently selected from O, N and S, where said chemical moiety is optionally substituted with one or more substituents selected from (C₁-C₄)alkyl, (C₁-C₄)alkoxy, trifluoromethyl, halo, cyano, amino, (C₁-C₄)alkyl amino, or di(C₁-C₄)alkyl amino;

or R⁸ taken together with R^{7a} form a 5- to 6-membered lactam;

(vi) —C(O)—O—R⁹, where R⁹ is (C₁-C₆)alkyl, halo-substituted (C₁-C₆)alkyl or (C₁-C₄)alkoxy(C₁-C₆)alkyl, or R⁹ taken together with R^{7a} form a 5- to 6-membered lactone;

(vii) —C(O)—N(R^{10a})(R^{10b}), where R^{10a} is hydrogen, (C₁-C₆)alkyl, or halo-substituted (C₁-C₄)alkyl, and R^{10b} is hydrogen, (C₁-C₆)alkyl, halo-substituted (C₁-C₄)alkyl, (C₂-C₆)alkenyl, (C₃-C₇)cycloalkyl, 3- to 6-membered heterocycle containing one to three heteroatoms independently selected from O, N and S, 5- to 6-membered lactam or lactone, and 5- to 6-membered heteroaryl containing one to three heteroatoms independently selected from O, N and S, where said chemical moiety is optionally substituted with one or more substituents selected from (C₁-C₄)alkyl, (C₁-C₄)alkoxy, trifluoromethyl, halo, cyano, amino, (C₁-C₄)alkyl amino, or di(C₁-C₄)alkyl amino,

or R^{10a} and R^{10b} taken together form a piperidine or pyrrolidine,

or either R^{10a} or R^{10b} taken together with R^{7a} form a 5- or 6-membered lactam;

a pharmaceutically acceptable salt thereof, or a solvate or hydrate of the compound or the salt.

Preferably, R¹ and R² are each independently a phenyl, where each phenyl is substituted with one or more substituents. More preferably, R¹ is a phenyl substituted with one to three substituents independently selected from the group consisting of halo (preferably, bromo, chloro or fluoro), (C₁-C₄)alkoxy, (C₁-C₄)alkyl, halo-substituted (C₁-C₄)alkyl (preferably fluoro-substituted alkyl, more preferably, trifluoromethyl) and cyano; and R² is a phenyl substituted with one to three substituents independently selected from the group consisting of halo (preferably, bromo, chloro or fluoro), (C₁-C₄)alkoxy, (C₁-C₄)alkyl, halo-substituted (C₁-C₄)alkyl (preferably fluoro-substituted alkyl, more preferably, trifluoromethyl) and cyano. Most preferably, R¹ is 2-chlorophenyl, 2-fluorophenyl, 2-bromophenyl, 2-cyanophenyl, 2,4-dichlorophenyl, 4-chloro-2-fluorophenyl, 2-chloro-4-fluorophenyl, 2-methylphenyl, 2-chloro-4-methylphenyl, or 2,4-difluorophenyl; and R² is 4-chlorophenyl, 4-cyanophenyl, 4-methylphenyl, 4-ethylphenyl, 4-isopropylphenyl, 4-methoxyphenyl, 4-ethoxyphenyl, 4-isopropoxyphenyl, 4-trifluoromethylphenyl, 4-fluorophenyl, and 4-bromophenyl.

Preferably, R^{3a}, R^{3b}, R⁴, R⁵, R^{5b}, R^{6a}, and R^{6b} are each hydrogen.

Preferred embodiments include any combination of the preferred substituents for R¹, R², V, W, R³, R^{3b}, R⁴, R⁵, R^{5b}, R^{6a}, R^{6b}, R^{7a} and/or R^{7b} with each other or with any or all of the original definitions for R¹, R², V, W, R³, R^{3b}, R⁴, R⁵, R^{5b}, R^{6a}, R^{6b}, R^{7a} and/or R^{7b}.

In one preferred embodiment of the present invention, a compound of Formula (II) is provided. 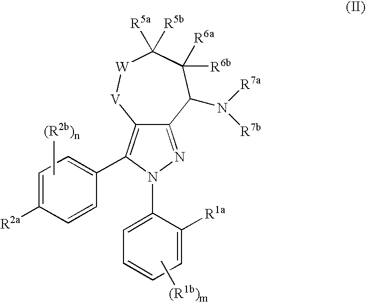
wherein

R^{1a}, R^{1b}, R^{2b}, and R^{2c} are each independently halo, (C₁-C₄)alkoxy, (C₁-C₄)alkyl, halo-substituted (C₁-C₄)alkyl, or cyano;

m and n are each independently 0, 1 or 2;

V, W, R^{3a}, R^{3b}, R⁴, R^{5a}, R^{5b}, R^{6a}, R^{6b}, R^{7a} and R^{7b} are as defined above for the comound of Formula (I);

a pharmaceutically acceptable salt thereof, or a solvate or hydrate of the compound or the salt.

Preferably, R^{1a} is chloro, fluoro, bromo, cyano, or methyl; m is 0 or 1; and R^{1b} is hydrogen (i.e., m is 0), chloro, fluoro, bromo, (C₁-C₄)alkyl, trifluoromethyl, (C₁-C₄)alkoxy, or cyano. Preferably, R^{2a} is chloro, fluoro, bromo, (C₁-C₄)alkyl, trifluoromethyl, (C₁-C₄)alkoxy, or cyano; and R^{2b} is hydrogen (i.e., n is 0). More preferred embodiments also include any combination of the preferred substituents for V, W, R^{3a}, R^{3b}, R⁴, R^{5a}, R^{5b}, R^{6a}, R^{6b}, R^{7a} and/or R^{7b} discussed above.

Another embodiment of the present invention includes a pharmaceutical composition comprising (1) a compound of the present invention, and (2) a pharmaceutically acceptable excipient, diluent, or carrier. Preferably, the composition comprises a therapeutically effective amount of a compound of the present invention. The composition may also contain at least one additional pharmaceutical agent (described herein). Preferred agents include nicotine receptor partial agonists, opioid antagonists (e.g., naltrexone and nalmefene), dopaminergic agents (e.g., apomorphine), attention deficit disorder (ADD including attentin deficit hyperactivity disorder (ADHD)) agents (e.g., Ritalin™, Strattera™, Concerta™ and Adderall™), and anti-obesity agents (described herein below).

Another embodiment of the present invention is a method for treating a disease, condition or disorder modulated by a cannabinoid receptor (preferably, a CB1 receptor) antagonists in animals that includes the step of administering to an animal in need of such treatment a therapeutically effective amount of a compound of the present invention (or a pharmaceutical composition thereof).

Diseases, conditions, and/or disorders modulated by cannabinoid receptor antagonists include those described herein below. In a preferred embodiment, the method is used in the treatment of obesity, attention deficit hyperactivity disorder, inflammation, dementia, alcoholism, and/or tobacco abuse.

Another embodiment of the present invention is a method for treating inflammatory pain or an inflammatory disease in animals that includes the step of administering to an animal in need of such treatment a therapeutically effective amount of a compound of the present invention (or a pharmaceutical composition thereof).

Another embodiment of the present invention is a method for treating arthritis, inflammatory bowel disease or congestive obstructive pulmonary disorder in animals that includes the step of administering to an animal in need of such treatment a therapeutically effective amount of a compound of the present invention (or a pharmaceutical composition thereof).

Compounds of the present invention may be administered in combination with other pharmaceutical agents. Preferred pharmaceutical agents include nicotine receptor partial agonists, opioid receptor antagonists (e.g., naltrexone (including naltrexone depot), antabuse, and nalmefene), dopaminergic agents (e.g., apomorphine), ADD/ADHD agents (e.g., methylphenidate hydrochloride (e.g., Ritalin™ and Concerta™), atomoxetine (e.g., Strattera™), and amphetamines (e.g., Adderall™)) and anti-obesity agents (described herein below).

The combination therapy may be administered as (a) a single pharmaceutical composition which comprises a compound of the present invention, at least one additional pharmaceutical agent described herein and a pharmaceutically acceptable excipient, diluent, or carrier; or (b) two separate pharmaceutical compositions comprising (i) a first composition comprising a compound of the present invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

### Definitions

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Similarly, the alkyl portion (i.e., alkyl moiety) of an alkoxy, acyl (e.g., alkanoyl), alkylamino, dialkylamino, and alkylthio group have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls) independently selected from the group of substituents listed below in the definition for “substituted.” “Halo-substituted alkyl” refers to an alkyl group substituted with one or more halogen atoms (e.g., “fluoro-substituted alkyl” refers to fluoromethyl, difluoromethyl, trifluoromethyl, 1-fluoroethyl, 2-fluoroethyl, 1,1-difluoroethyl, 1,2-difluoroethyl, 2,2-difluoroethyl, 1,1,1-trifluoroethyl, 2,2,2-trifluoroethyl, 1,1,2-trifluoroethyl, 1,2,2-trifluoroethyl, 1,2,2,2-tetrafluoroethyl, 1,1,2,2-tetrafluoroethyl, 1,1,1,2-tetrafluoroethyl, 1,1,2,2,2-pentafluoroethyl, 1,1,1,2,2-pentafluoroethyl, perfluoroethyl, etc.). Preferred halo-substituted alkyls are the chloro- and fluoro-substituted alkyls, more preferably, fluoro-substituted alkyls.

When substituted, the alkane radicals or alkyl moieties are preferably substituted with 1 to 3 fluoro substituents, or 1 or 2 substituents independently selected from (C₁-C₃)alkyl, (C₃-C₆)cycloalkyl, (C₂-C₃)alkenyl, aryl, heteroaryl, 3- to 6-membered heterocycle, chloro, cyano, hydroxy, (C₁-C₃)alkoxy, aryloxy (e.g., phenoxy), amino, (C₁-C₆)alkyl amino, di-(C₁-C₄)alkyl amino, aminocarboxylate (i.e., (C₁-C₃)alkyl-O—C(O)—NH—), hydroxy(C₂-C₃)alkylamino, or keto (oxo), and more preferably, 1 to 3 fluoro groups, or 1 substituent selected from (C₁-C₃)alkyl, (C₃-C₆)cycloalkyl, phenyl, 6-membered-heteroaryl, 3- to 6-membered heterocycle, (C₁-C₃)alkoxy, (C₁-C₄)alkylamino or di-(C₁-C₂)alkyl amino.

The terms “partially or fully saturated carbocyclic ring” (also referred to as “partially or fully saturated cycloalkyl”) refers to nonaromatic rings that are either partially or fully hydrogenated and may exist as a single ring, bicyclic ring or a spiral ring. Unless specified otherwise, the carbocyclic ring is generally a 3- to 8-membered ring. For example, partially or fully saturated carbocyclic rings (or cycloalkyl) include groups such as cyclopropyl, cyclopropenyl, cyclobutyl, cyclobutenyl, cyclopentyl, cyclpentenyl, cyclopentadienyl, cyclohexyl, cyclohexenyl, cyclohexadienyl, norbornyl (bicyclo[2.2.1]heptyl), norbornenyl, bicyclo[2.2.2]octyl, and the like. When designated as being “optionally substituted”, the partially saturated or fully saturated cycloalkyl group may be unsubstituted or substituted with one or more substituents (typically, one to three substituents) independently selected from the group of substituents listed below in the definition for “substituted.” A substituted carbocyclic ring also includes groups wherein the carbocyclic ring is fused to a phenyl ring (e.g., indanyl). The carbocyclic group may be attached to the chemical entity or moiety by any one of the carbon atoms within the carbocyclic ring system. When substituted, the carbocyclic group is preferably substituted with 1 or 2 substituents independently selected from (C₁-C₃)alkyl, (C₂-C₃)alkenyl, (C₁-C₆)alkylidenyl, aryl, heteroaryl, 3- to 6-membered heterocycle, chloro, fluoro, cyano, hydroxy, (C₁-C₃)alkoxy, aryloxy, amino, (C₁-C₆)alkyl amino, di-(C₁-C₄)alkyl amino, aminocarboxylate (i.e., (C₁-C₃)alkyl-O—C(O)—NH—), hydroxy(C₂-C₃)alkylamino, or keto (oxo), and more preferably 1 or 2 from substituents independently selected from (C₁-C₂)alkyl, 3- to 6-membered heterocycle, fluoro, (C₁-C₃)alkoxy, (C₁-C₄)alkyl amino or di-(C₁-C₂)alkyl amino. Similarly, any cycloalkyl portion of a group (e.g., cycloalkylalkyl, cycloalkylamino, etc.) has the same definition as above.

The term “partially saturated or fully saturated heterocyclic ring” (also referred to as “partially saturated or fully saturated heterocycle”) refers to nonaromatic rings that are either partially or fully hydrogenated and may exist as a single ring, bicyclic ring or a spiral ring. Unless specified otherwise, the heterocyclic ring is generally a 3- to 6-membered ring containing 1 to 3 heteroatoms (preferably 1 or 2 heteroatoms) independently selected from sulfur, oxygen and/or nitrogen. Partially saturated or fully saturated heterocyclic rings include groups such as epoxy, aziridinyl, tetrahydrofuranyl, dihydrofuranyl, dihydropyridinyl, pyrrolidinyl, N-methylpyrrolidinyl, imidazolidinyl, imidazolinyl, piperidinyl, piperazinyl, pyrazolidinyl, 2H-pyranyl, 4H-pyranyl, 2H-chromenyl, oxazinyl, morpholino, thiomorpholino, tetrahydrothienyl, tetrahydrothienyl 1,1-dioxide, and the like. When indicated as being “optionally substituted”, the partially saturated or fully saturated heterocycle group may be unsubstiuted or substituted with one or more substituents (typically, one to three substituents) independently selected from the group of substituents listed below in the definition for “substituted.” A substituted heterocyclic ring includes groups wherein the heterocyclic ring is fused to an aryl or heteroaryl ring (e.g., 2,3-dihydrobenzofuranyl, 2,3-dihydroindolyl, 2,3-dihydrobenzothiophenyl, 2,3-dihydrobenzothiazolyl, etc.). When substituted, the heterocycle group is preferably substituted with 1 or 2 substituents independently selected from (C₁-C₃)alkyl, (C₃-C₆)cycloalkyl, (C₂-C₄)alkenyl, aryl, heteroaryl, 3- to 6-membered heterocycle, chloro, fluoro, cyano, hydroxy, (C₁-C₃)alkoxy, aryloxy, amino, (C₁-C₆)alkyl amino, di-(C₁-C₃)alkyl amino, aminocarboxylate (i.e., (C₁-C₃)alkyl-O—C(O)—NH—), or keto (oxo), and more preferably with 1 or 2 substituents independently selected from (C₁-C₃)alkyl, (C₃-C₆)cycloalkyl, (C₆)aryl, 6-membered-heteroaryl, 3- to 6-membered heterocycle, or fluoro. The heterocyclic group may be attached to the chemical entity or moiety by any one of the ring atoms within the heterocyclic ring system. Similarly, any heterocycle portion of a group (e.g., heterocycle-substituted alkyl, heterocycle carbonyl, etc.) has the same definition as above.

The term “aryl” or “aromatic carbocyclic ring” refers to aromatic moieties having a single (e.g., phenyl) or a fused ring system (e.g., naphthalene, anthracene, phenanthrene, etc.). A typical aryl group is a 6- to 10-membered aromatic carbocyclic ring(s). When indicated as being “optionally substituted”, the aryl groups may be unsubstituted or substituted with one or more substituents (preferably no more than three substituents) independently selected from the group of substituents listed below in the definition for “substituted.” Substituted aryl groups include a chain of aromatic moieties (e.g., biphenyl, terphenyl, phenylnaphthalyl, etc.). When substituted, the aromatic moieties are preferably substituted with 1 or 2 substituents independently selected from (C₁-C₄)alkyl, (C₂-C₃)alkenyl, aryl, heteroaryl, 3- to 6-membered heterocycle, bromo, chloro, fluoro, iodo, cyano, hydroxy, (C₁-C₄)alkoxy, aryloxy, amino, (C₁-C₆)alkyl amino, di-(C₁-C₃)alkyl amino, or aminocarboxylate (i.e., (C₁-C₃)alkyl-O—C(O)—NH—), and more preferably, 1 or 2 substituents independently selected from (C₁-C₄)alkyl, chloro, fluoro, cyano, hydroxy, or (C₁-C₄)alkoxy. The aryl group may be attached to the chemical entity or moiety by any one of the carbon atoms within the aromatic ring system. Similarly, the aryl portion (i.e., aromatic moiety) of an aroyl or aroyloxy (i.e., (aryl)-C(O)—O—) has the same definition as above.

The term “heteroaryl” or “heteroaromatic ring” refers to aromatic moieties containing at least one heteratom (e.g., oxygen, sulfur, nitrogen or combinations thereof) within a 5- to 10-membered aromatic ring system (e.g., pyrrolyl, pyridyl, pyrazolyl, indolyl, indazolyl, thienyl, furanyl, benzofuranyl, oxazolyl, imidazolyl, tetrazolyl, triazinyl, pyrimidyl, pyrazinyl, thiazolyl, purinyl, benzimidazolyl, quinolinyl, isoquinolinyl, benzothiophenyl, benzoxazolyl, etc.). The heteroaromatic moiety may consist of a single or fused ring system. A typical single heteroaryl ring is a 5- to 6-membered ring containing one to three heteroatoms independently selected from oxygen, sulfur and nitrogen and a typical fused heteroaryl ring system is a 9- to 10-membered ring system containing one to four heteroatoms independently selected from oxygen, sulfur and nitrogen. When indicated as being “optionally substituted”, the heteroaryl groups may be unsubstituted or substituted with one or more substituents (preferably no more than three substituents) independently selected from the group of substituents listed below in the definition for “substituted.” When substituted, the heteroaromatic moieties are preferably substituted with 1 or 2 substituents independently selected from (C₁-C₄)alkyl, (C₂-C₃)alkenyl, aryl, heteroaryl, 3- to 6-membered heterocycle, bromo, chloro, fluoro, iodo, cyano, hydroxy, (C₁-C₄)alkoxy, aryloxy, amino, (C₁-C₆)alkyl amino, di-(C₁-C₃)alkyl amino, or aminocarboxylate (i.e., (C₁-C₃)alkyl-O—C(O)—NH—), and more preferably, 1 or 2 substituents independently selected from (C₁-C₄)alkyl, chloro, fluoro, cyano, hydroxy, (C₁-C₄)alkoxy, (C₁-C₄)alkyl amino or di-(C₁-C₂)alkyl amino. The heteroaryl group may be attached to the chemical entity or moiety by any one of the atoms within the aromatic ring system (e.g., imidazol-1-yl, imidazol-2-yl, imidazol-4-yl, imidazol-5-yl, pyrid-2-yl, pyrid-3-yl, pyrid-4-yl, pyrid-5-yl, or pyrid-6-yl). Similarly, the heteroaryl portion (i.e., heteroaromatic moiety) of a heteroaroyl or heteroaryoloxy (i.e., (heteroaryl)-C(O)—O—) has the same definition as above.

The term “acyl” refers to hydrogen, alkyl, partially saturated or fully saturated cycloalkyl, partially saturated or fully saturated heterocycle, aryl, and heteroaryl substituted carbonyl groups. For example, acyl includes groups such as (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions above. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “substituted” specifically envisions and allows for one or more substitutions that are common in the art. However, it is generally understood by those skilled in the art that the substituents should be selected so as to not adversely affect the pharmacological characteristics of the compound or adversely interfere with the use of the medicament. Suitable substituents for any of the groups defined above include (C₁-C₆)alkyl, (C₃-C₇)cycloalkyl, (C₂-C₆)alkenyl, (C₁-C₆)alkylidenyl, aryl, heteroaryl, 3- to 6-membered heterocycle, halo (e.g., chloro, bromo, iodo and fluoro), cyano, hydroxy, (C₁-C₆)alkoxy, aryloxy, sulfhydryl (mercapto), (C₁-C₆)alkylthio, arylthio, amino, mono- or di-(C₁-C₆)alkyl amino, quaternary ammonium salts, amino(C₁-C₆)alkoxy, aminocarboxylate (i.e., (C₁-C₆)alkyl-O—C(O)—NH—), hydroxy(C₂-C₆)alkylamino, amino(C₁-C₆)alkylthio, cyanoamino, nitro, (C₁-C₆)carbamyl, keto (oxo), acyl, (C₁-C₆)alkyl-CO₂—, glycolyl, glycyl, hydrazino, guanyl, sulfamyl, sulfonyl, sulfinyl, thio(C₁-C₆)alkyl-C(O)—, thio(C₁-C₆)alkyl-CO₂—, and combinations thereof. In the case of substituted combinations, such as “substituted aryl(C₁-C₆)alkyl”, either the aryl or the alkyl group may be substituted, or both the aryl and the alkyl groups may be substituted with one or more substituents (typically, one to three substituents except in the case of perhalo substitutions). An aryl or heteroaryl substituted carbocyclic or heterocyclic group may be a fused ring (e.g., indanyl, dihydrobenzofuranyl, dihydroindolyl, etc.).

The term “solvate” refers to a molecular complex of a compound represented by Formula (I) or (II) (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The terms “modulated by a cannabinoid receptor” or “modulation of a cannabinoid receptor” refers to the activation or deactivation of a cannabinoid receptor. For example, a ligand may act as an agonist, partial agonist, inverse agonist, antagonist, or partial antagonist.

The term “antagonist” includes both full antagonists and partial antagonists, as well as inverse agonists.

The term “CB-1 receptor” refers to the G-protein coupled type 1 cannabinoid receptor.

The term “compounds of the present invention” (unless specifically identified otherwise) refer to compounds of Formulae (I) and (II), pharmaceutically acceptable salts of the compounds, and hydrates or solvates of the compounds, and/or salts, as well as, all stereoisomers (including diastereoisomers and enantiomers), tautomers and isotopically labeled compounds.

### DETAILED DESCRIPTION

The present invention provides compounds and pharmaceutical formulations thereof that are useful in the treatment of diseases, conditions and/or disorders modulated by cannabinoid receptor antagonists.

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Procedures analogous to those described in U.S. Patent Publication No. 2004/0214855 and U.S. patent application Ser. No. 10/971,599 entitled “Bicyclic Pyrazolyl And Imidazolyl Compounds and Uses Thereof” filed on Oct. 22, 2004, both of which are incorporated herein by reference, are useful for preparing key intermediates that may be used in the preparation of the compounds of the present invention.

Scheme I outlines the procedures one could use to provide compounds of the present invention where V is oxygen, W is CR^{3a}R^{3b}, R^{5a}, R^{5b}, R^{6a} and R^{6b} are each independently hydrogen, (C₁-C₄)alkyl, or halo-substituted (C₁-C₄)alkyl, and R^{7a} and R^{7b} are each independently hydrogen, (C₁-C₆)alkyl, (C₂-C₆)alkenyl, or halo-substituted (C₁-C₄)alkyl.

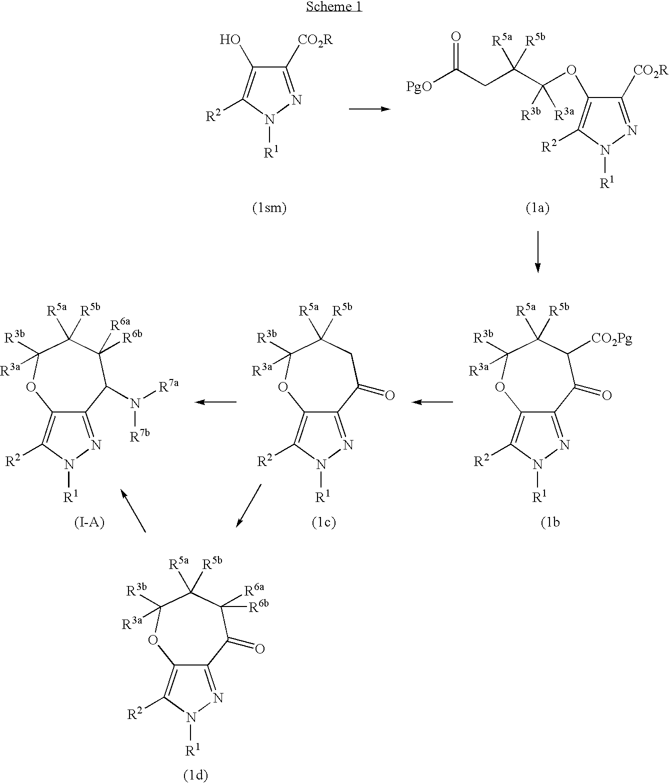

The starting material (1 sm) may be prepared as described in U.S. patent application Ser. No. 10/971,599 entitled “Bicyclic Pyrazolyl and Imidazolyl Compounds and Uses Thereof” filed on Oct. 22, 2004 and U.S. Provisional Patent Application Ser. No. 60/613,613 filed on Sep. 27, 2004, both of which are incorporated herein by reference. See also the Example section below for a representative preparation.

The hydroxy ester starting material (1 sm, where R is an alkyl group) is condensed with the desired carboxy-protected carboxylic acid containing a leaving group (e.g., tert-butyl 4-bromobutanoate) in the presence of a non-nucleophilic base (e.g., potassium tert-butoxide) at cooled temperatures (e.g., about 0° C.) in a polar aprotic solvent (e.g., dimethylformamide (DMF)) to provide intermediate (1a). The cyclized intermediate (1c) may then be produced by treating intermediate (1a) with a suitable base (e.g., potassium hexamethyldisilazane (KHMDS)) at reduced temperatures (e.g., about −78° C. to about 0° C.) in a non-polar solvent (e.g., tetrahydrofuran (THF)). The carboxy-protecting group is first removed by treating with a strong acid (e.g., trifluoroacetic acid) at or near room temperature and then the resultant carboxylic acid group is decarboxylated by heating at elevated temperatures (e.g., refluxing 1:1 toluene:dioxane). The ketone intermediate (1c) may then be treated with sodium cyanoborohydride and ammonium acetate to form the amino compound (1-A, where R^{7a} and R^{7b} are both hydrogen). Alternatively, the ketone intermediate (1c) may be treated with the desired alkyl or halo-alkyl substituted amine and sodium triacetoxyborohydride to produce an amino compound I-A, where R^{7a} or R^{7b} is (C₁-C₆)alkyl, (C₂-C₆)alkenyl, or halo-substituted (C₁-C₄)alkyl. For those compounds having a substituent R^{6a} and/or R^{6b}, ketone intermediate (1c) may be alklated with a desired alkylating agent in the presence of a base to produce intermediate (1d) prior to the introduction of the amino group.

Scheme II below outlines the procedures one could use to provide compounds of the present invention where V is oxygen, and R^{7b} is —C(O)—R⁸.

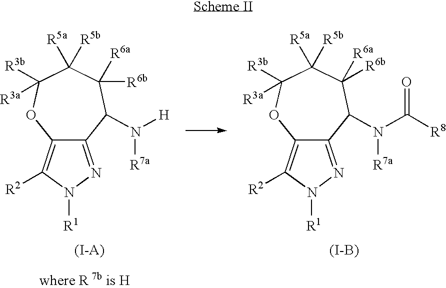

The amino compound (I-A) may be converted to the corresponding amide (I-B) by treating the amino compound I-A (where the amino group is either a primary or secondary amine) with the desired acylating agent (e.g., acyl chloride, such as Cl—C(O)—R⁸) in the presence of a base (e.g., triethylamine).

Scheme III below outlines the procedures one could use to provide compounds of the present invention where V is oxygen, W is CR^{3a}R^{3b} and R^{7b} is —C(O)—O—R⁹.

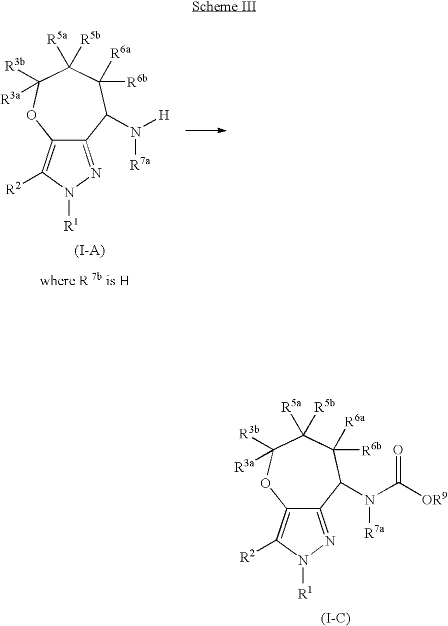

The amino compound (I-A) may be converted to the corresponding carbamate (I-C) by treating the amino compound I-A (where the amino group is either a primary or secondary amine) with the desired chloroformate (i.e., Cl—C(O)—OR⁹) in the presence of a base (e.g., triethylamine).

Scheme IV below outlines the procedures one could use to provide compounds of the present invention where V is oxygen, and R^{7b} is —C(O)—N(R^{10a})(R^{10b}), where R^{10b} is hydrogen.

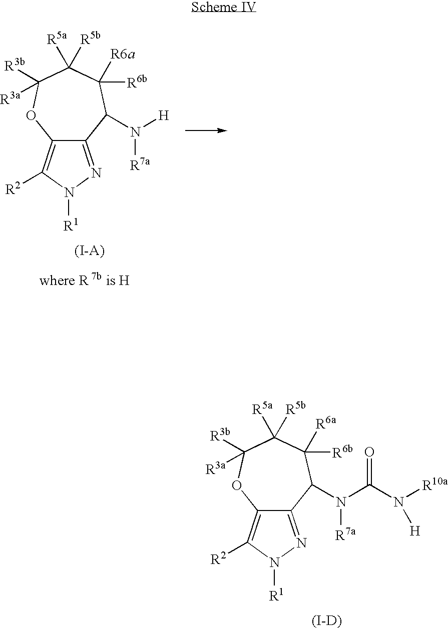

The amino compound (I-A) may be converted to the corresponding urea (I-D) by treating the amino compound I-A (where the amino group is either a primary or secondary amine) with the desired isocyanate (R^{10a}—N═C═O).

Scheme V below outlines the procedures one could use to provide compounds of the present invention where V is oxygen, and R^{7b} is —C(O)—N(R^{10a})(R^{10b}), where neither R^{10a} nor R^{10b} are hydrogen

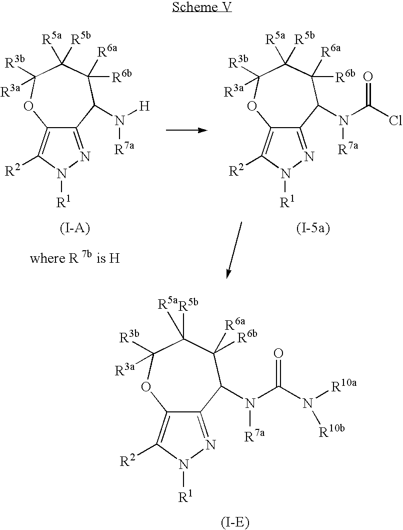

The carbamoyl chloride intermediate (I-5a) may be prepared by reacting compound I-A with dichloroformate in the presence of a hindered amine (e.g., triethylamine). The resultant carbamoyl chloride intermediate (I-5a) may then be reacted with the desired amine (HNR^{10a}R^{10b}) to produce the compound of Formula I-E. Alternatively, the compound of Formula I-E may be prepared from the carbamate I-C (see, Scheme III above) by either heating directly with the desired amine or in the presence of trimethyl aluminum as described by Lee, S-H, et al., in Tetrahedron, 60(15), 3439-3443 (2004).

Scheme VI outlines the procedures one could use to provide compounds of the present invention such as I-F, I-G, and I-H where V is carbon, W is nitrogen, R⁴ is hydrogen.

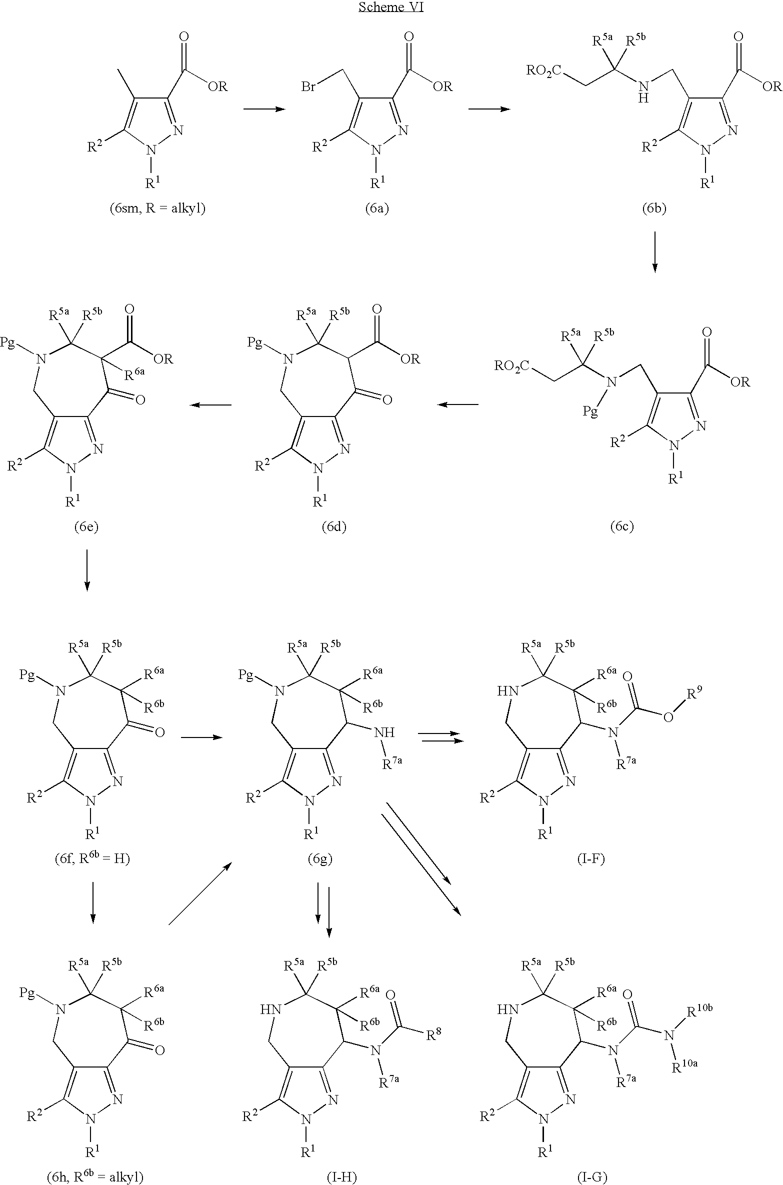

Starting material (9sm) may be prepared using procedures described by Barth, et al., in European Application No. 656354 and then brominated using using procedures analogous to those described by Barth, et al., in PCT Publication No. WO97/19063. For example, starting material (6sm) is treated with 2,2′-azobisisobutyronitrile (AIBN) in carbon tetrachloride at elevated temperatures (e.g., reflux) to give (6a). Alkylation of (6a) with an appropriately substituted □-alanine derivative (e.g., H₂NC(R⁴)(R^{4b})CH₂CO₂R) in the presence of a base (e.g., K₂CO₃, Na₂CO₃) may provide the amino derivative (6b) which may be converted to the N-protected derivative (6c) by methods known in the art. Compound (6c) may be reacted with a base (e.g., alkali metal alkoxide such as sodium ethoxide, sodium methoxide, or potassium tert.-butoxide) in an alcoholic solvent (e.g., EtOH, MeOH, tert.-BuOH) at elevated temperatures (e.g., reflux) to give the tricyclic product (6d). Reaction of (6d) with R^{6a}—X (where X is a leaving group) in the presence of a base (e.g., K₂CO₃) in a suitable solvent (e.g., THF, DMF) may provide (6e). The carboxylate ester group in (6e) may be removed by treating the compound with NaCl in a suitable solvent (e.g., aqueous DMSO) using procedures as described in Tetrahedron, 45(21), 6833-6840 (1989). The ketone intermediate (6f) may then be treated with the sodium cyanoborohydride and ammonium acetate to form the amino compound (6g, where R^{7a} and R^{7b} are both hydrogen). Alternatively, the ketone intermediate (6f) may be treated with the desired alkyl or halo-alkyl substituted amine to produce an amino compound (6g), where R⁷ or R^{7b} is (C₁-C₆)alkyl, (C₂-C₆)alkenyl, or halo-substituted (C₁-C₄)alkyl. For those compounds having a substituent R^{6b}, ketone intermediate (6f) may be treated first with a desired alkylating agent (R^{6b}—X) in the presence of a base (NaH, KHMDS, LiHMDS) in a polar, aprotic solvent such as THF and then with the desired alkyl or halo-alkyl substituted amine. Compound (6g) may be converted into compounds of formula I-F, I-G, and I-H using the general methods described in Schemes II-IV and an additional step that involves removal of the protecting group.

An alternate method to synthesize intermediate (6c) is described in Scheme VII.

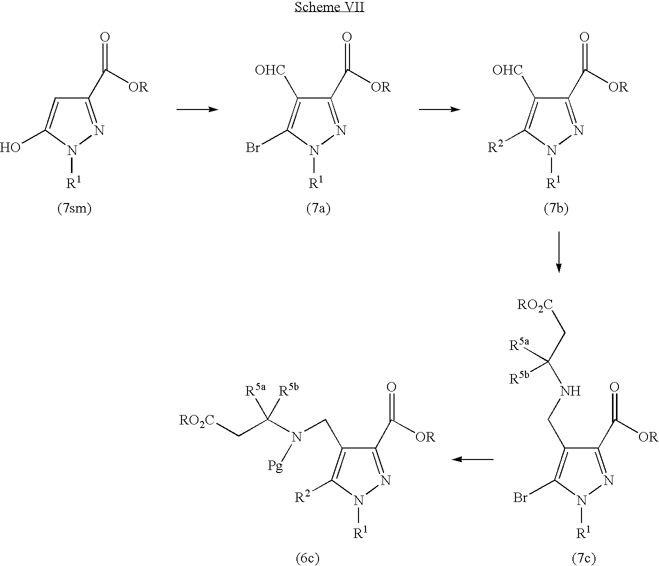

Starting material (7sm), prepared as described in U.S. Patent Publication No. 2004248881, may be converted into the bromo aldehyde derivative (7a) using phosphorus oxybromide (POBr₃) in a solvent such as DMF at elevated temperatures (e.g, refluxing). The R² group may be introduced into (7a) as described in U.S. Patent Publication No. 20040214855 by displacing the bromo group on the pyrazolyl ring with the desired R² group. This may be accomplished by treating intermediate (7a) with either the desired boronic acid (R²—B(OH)₂) or tin reagent (R²SnR₃) in the presence of cesium fluoride and tetrakis(triphenylphosphine)palladium(0) in a polar solvent (e.g., 1,2-dimethoxyethane) at elevated temperatures (e.g., 100° C.) to give intermediate (7b). The formyl group in derivative (7b) may be reacted with a substituted □-alanine derivative (e.g., H₂NC(R^{4a})(R^{4b})CH₂CO₂R) in the presence of a reducing agent (e.g., sodium borohydride, sodium triacetoxyborohydride) and a weak acid (e.g., acetic acid) to give (7c). The amino group in (7b) may be protected with a suitable group (e.g., BOC, Bn) to provide (6c).

Scheme VII below outlines the procedures one could use to provide compounds of the present invention where R⁴ is alkyl, alkylcarbonyl, arylcarbonyl, alkylsulfonyl and arylsulfonyl.

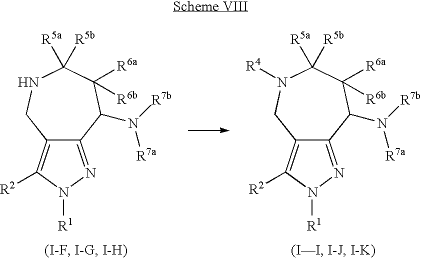

Compounds (I-F, I-G, I-H) may be alkylated with R⁴X (where X is a leaving group) in a polar, aprotic solvent (e.g., DMF or THF) to provide compounds (I-I, I-J, I-K). Compounds (I-F, I-G, I-H) may also be reacted with an aldehyde or ketone derivative in the presence of a reducing agent such as NaBH(OAc)₃ in a solvent such as dichloroethane to produce compounds of formula I. Compounds (I-F, I-G, I-H) may be treated with acid chlorides or sulfonyl chlorides and a base (e.g., triethylamine) in the presence of a catalytic amount of DMAP in a non-polar solvent such as CH₂Cl₂ to give compounds where R⁴ is alkyl, alkylcarbonyl, arylcarbonyl, alkylsulfonyl and arylsulfonyl.

Conventional methods and/or techniques of separation and purification known to one of ordinary skill in the art can be used to isolate the compounds of the present invention, as well as the various intermediates related thereto. Such techniques will be well-known to one of ordinary skill in the art and may include, for example, all types of chromatography (high pressure liquid chromatography (HPLC), column chromatography using common adsorbents such as silica gel, and thin-layer chromatography), recrystallization, and differential (i.e., liquid-liquid) extraction techniques.

The compounds of the present invention may be isolated and used per se or in the form of its pharmaceutically acceptable salt, solvate and/or hydrate. The term “salts” refers to inorganic and organic salts of a compound of the present invention. These salts can be prepared in situ during the final isolation and purification of a compound, or by separately reacting the compound, N-oxide, or prodrug with a suitable organic or inorganic acid or base and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

A prodrug may also be utilized to provide an alternative means of introducing the compound of the present invention in therapeutic use. The term “prodrug” means a compound that is transformed in vivo to yield a compound of Formula (I) or a pharmaceutically acceptable salt, hydrate or solvate of the compound. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987. Prodrugs are also known as “esters” as defined by the United States Federal Drug Administration.

For example, if a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound of the present invention contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, x-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, P(O)(O(C₁-C₆)alkyl)₂ or glycosyl (the radical resulting from the removal of a hydroxyl group of the hemiacetal form of a carbohydrate).

If a compound of the present invention incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY′ wherein Y′ is H, (C₁-C₆)alkyl or benzyl, —C(OY₀)Y₁ wherein Y₀ is (C₁-C₄) alkyl and Y₁ is (C₁-C₆)alkyl, carboxy(C₁-C₆)alkyl, amino(C₁-C₄)alkyl or mono-N— or di-N,N-(C₁-C₆)alkylaminoalkyl, —C(Y₂)Y₃ wherein Y₂ is H or methyl and Y₃ is mono-N— or di-N,N-(C₁-C₆)alkylamino, morpholino, piperidin-1-yl or pyrrolidin-1-yl.

The compounds of the present invention may contain asymmetric or chiral centers, and, therefore, exist in different stereoisomeric forms. It is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans-forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

The compounds of the present invention may exist in unsolvated as well as solvated forms with pharmaceutically acceptable solvents (e.g., Class 3 solvents, including water, ethanol, and the like). Class 3 solvents are listed in the United States Food and Drug Administration's Guidance for Industry, Q3C Tables and Lists. (Copies are available from the Center for Drug Evaluation and Research (CDER) Division of Drug Information (HFD-240) Food and Drug Administration, 5600 Fishers Lane, Rockville, Md., USA, 20857 or the internet at http://www.fda.gov/cder/quidance/index.htm). It is intended that the invention embrace both solvated and unsolvated forms.

It is also possible that the compounds of the present invention may exist in different tautomeric forms, and all such forms are embraced within the scope of the invention. For example, all of the tautomeric forms of the aromatic and heteroaromatic moieties are included in the invention. Also, for example, all keto-enol and imine-enamine forms of the compounds are included in the invention.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine, iodine, and chlorine, such as ²H, ³H, ¹³C, ¹⁴C, ¹⁵N, ¹⁸O, ¹⁷O, ³¹P, ³²P, ³⁵S, ¹⁸F, ¹²³I, and ³⁶Cl, respectively.

Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Schemes and/or in the Examples hereinbelow, by substituting an isotopically labeled reagent for a non-isotopically labeled reagent.

Compounds of the present invention are useful for treating diseases, conditions and/or disorders modulated by cannabinoid receptor antagonists; therefore, another embodiment of the present invention is a method of treating diseases, conditions and/or disorders modulated by cannabinoid receptor antagonists in an animal that includes administering to an animal in need of such treatment a therapeutically effective amount of a compound of the present invention or a pharmaceutical composition comprising an effective amount of a compound of the present invention and a pharmaceutically acceptable excipient, diluent, or carrier. Consequently, the compounds of the present invention (including the compositions and processes used therein) may be used in the manufacture of a medicament for the therapeutic applications described herein.

Preliminary investigations have indicated that the following diseases, conditions, and/or disorders are modulated by cannabinoid receptor antagonists: eating disorders (e.g., binge eating disorder, anorexia, and bulimia), weight loss or control (e.g., reduction in calorie or food intake, and/or appetite suppression), obesity, depression, atypical depression, bipolar disorders, psychoses, schizophrenia, behavioral addictions, suppression of reward-related behaviors (e.g., conditioned place avoidance, such as suppression of cocaine- and morphine-induced conditioned place preference), substance abuse, addictive disorders, impulsivity, alcoholism (e.g., alcohol abuse, addiction and/or dependence including treatment for abstinence, craving reduction and relapse prevention of alcohol intake), tobacco abuse (e.g., smoking addiction, cessation and/or dependence including treatment for craving reduction and relapse prevention of tobacco smoking), dementia (including memory loss, Alzheimer's disease, dementia of aging, vascular dementia, mild cognitive impairment, age-related cognitive decline, and mild neurocognitive disorder), sexual dysfunction in males (e.g., erectile difficulty), seizure disorders, epilepsy, inflammation, gastrointestinal disorders (e.g., dysfunction of gastrointestinal motility or intestinal propulsion), attention deficit disorder (including ADHD), Parkinson's disease, and type II diabetes.

The present invention provides a method for treating inflammatory diseases comprising the step of administering to an animal (preferably, a mammal, more preferably a human) in need thereof a therapeutically effective amount of a compound of the present invention. Preferred inflammatory diseases include arthritis, inflammatory bowel disease and congestive obstructive pulmonary disorder. Preferably, the therapeutically effective amount is an amount sufficient to decrease the concentration of TNFα or MIP-1α and/or increase the concentration of IL-10 in the blood serum of the animal. The reduction of TNFα and/or MIP-1α that is significant or desirable is 40-60% (preferred), 60-80% (more preferred) and 80-100% (most preferred). In contrast, the increase in IL-10 that is significant or desirable is 10-35% (preferred), 35-70% (more preferred) and 75-100% (most preferred). Preferably, the therapeutically effective amount is an amount sufficient to decrease the concentration of TNFα or MIP-1α and/or increase the concentration of IL-10 in the blood serum of the animal. The reduction of TNFα and/or MIP-1α that is significant or desirable is 40-60% (preferred), 60-80% (more preferred) and 80-100% (most preferred). In contrast, the increase in IL-10 that is significant or desirable is 10-35% (preferred), 35-70% (more preferred) and 75-100% (most preferred).

The present invention also provides a method for reducing the symptoms of inflammation (e.g., swelling) comprising the step of administering to an animal (preferably a mammal, more preferably a human) in need thereof a therapeutically effective amount of a compound of the present invention. Preferably, the therapeutically effective amount is an amount sufficient to inhibit production of PGE2 and TNF α. The reduction of TNF α or PGE2 that is significant or desirable is 40-60% (preferred), 60-80% (more preferred) and 80-100% (most preferred).

The present invention also provides a method for treating inflammatory pain comprising the step of administering to an animal (preferably, a mammal, more preferably, a human) in need thereof a therapeutically effective amount of a compound of the present invention.

The present invention also provides a method for treating arthritis (preferably, rheumatoid arthritis) comprising the step of administering to an animal (preferably, a mammal, more preferably, a human) in need thereof a therapeutically effective amount of a compound of Formula I.

Other diseases, conditions and/or disorders for which cannabinoid receptor antagonists may be effective include: premenstrual syndrome or late luteal phase syndrome, migraines, panic disorder, anxiety, post-traumatic syndrome, social phobia, cognitive impairment in non-demented individuals, non-amnestic mild cognitive impairment, post operative cognitive decline, disorders associated with impulsive behaviours (such as, disruptive behaviour disorders (e.g., anxiety/depression, executive function improvement, tic disorders, conduct disorder and/or oppositional defiant disorder), adult personality disorders (e.g., borderline personality disorder and antisocial personality disorder), diseases associated with impulsive behaviours (e.g., substance abuse, paraphilias and self-mutilation), and impulse control disorders (e.g., intermittene explosive disorder, kleptomania, pyromania, pathological gambling, and trichotillomania)), obsessive compulsive disorder, chronic fatigue syndrome, sexual dysfunction in males (e.g., premature ejaculation), sexual dysfunction in females, disorders of sleep (e.g., sleep apnea), autism, mutism, neurodengenerative movement disorders, spinal cord injury, damage of the central nervous system (e.g., trauma), stroke, neurodegenerative diseases or toxic or infective CNS diseases (e.g., encephalitis or meningitis), cardiovascular disorders (e.g., thrombosis), and diabetes.

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents such as apolipoprotein-B secretion/microsomal triglyceride transfer protein (apo-B/MTP) inhibitors, 11β-hydroxy steroid dehydrogenase-1 (11β-HSD type 1) inhibitors, peptide YY₃₋₃₆ or analogs thereof, MCR-4 receptor agonists, cholecystokinin-A (CCK-A) agonists, monoamine reuptake inhibitors (such as sibutramine), sympathomimetic agents, β₃ adrenergic receptor agonists, dopamine receptor agonists (such as bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y receptor antagonists (e.g., NPY Y5 receptor antagonists, such as the spiro compounds described in U.S. Pat. Nos. 6,566,367; 6,649,624; 6,638,942; 6,605,720; 6,495,559; 6,462,053; 6,388,077; 6,335,345; and 6,326,375; US Publication Nos. 2002/0151456 and 2003/036652; and PCT Publication Nos. WO 03/010175. WO 03/082190 and WO 02/048152), thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or inverse agonists, neuromedin U receptor agonists and the like. Other anti-obesity agents, including the preferred agents set forth hereinbelow, are well known, or will be readily apparent in light of the instant disclosure, to one of ordinary skill in the art.

Especially preferred are anti-obesity agents selected from the group consisting of orlistat, sibutramine, bromocriptine, ephedrine, leptin, pseudoephedrine; peptide YY₃₋₃₆ or an analog thereof; and 2-oxo-N-(5-phenylpyrazinyl)spiro-[isobenzofuran-1(3H),4′-piperidine]-1′-carboxamide. Preferably, compounds of the present invention and combination therapies are administered in conjunction with exercise and a sensible diet.

Representative anti-obesity agents for use in the combinations, pharmaceutical compositions, and methods of the invention can be prepared using methods known to one of ordinary skill in the art, for example, sibutramine can be prepared as described in U.S. Pat. No. 4,929,629; bromocriptine can be prepared as described in U.S. Pat. Nos. 3,752,814 and 3,752,888; orlistat can be prepared as described in U.S. Pat. Nos. 5,274,143; 5,420,305; 5,540,917; and 5,643,874; PYY₃₋₃₆ (including analogs) can be prepared as described in US Publication No. 2002/0141985 and WO 03/027637; and the NPY Y5 receptor antagonist 2-oxo-N-(5-phenylpyrazinyl)spiro[isobenzofuran-1(3H),4′-piperidine]-1′-carboxamide can be prepared as described in US Publication No. 2002/0151456. Other useful NPY Y5 receptor antagonists include those described in PCT Publication No. 03/082190, such as 3-oxo-N-(5-phenyl-2-pyrazinyl)-spiro[isobenzofuran-1(3H),4′-piperidine]-1′-carboxamide; 3-oxo-N-(7-trifluoromethylpyrido[3,2-b]pyridin-2-yl)-spiro-[isobenzofuran-1(3H),4′-piperidine]-1′-carboxamide; N-[5-(3-fluorophenyl)-2-pyrimidinyl]-3-oxospiro-[isobenzofuran-1(3H),[4′-piperidine]-1′-carboxamide; trans-3′-oxo-N-(5-phenyl-2-pyrimidinyl)]spiro[cyclohexane-1,1′(3′H)-isobenzofuran]-4-carboxamide; trans-3′-oxo-N-[1-(3-quinolyl)-4-imidazolyl]spiro[cyclohexane-1,1′(3′H)-isobenzofuran]-4-carboxamide; trans-3-oxo-N-(5-phenyl-2-pyrazinyl)spiro[4-azaiso-benzofuran-1(3H),1′-cyclohexane]-4′-carboxamide; trans-N-[5-(3-fluorophenyl)-2-pyrimidinyl]-3-oxospiro[5-azaisobenzofuran-1(3H),1′-cyclohexane]-4′-carboxamide; trans-N-[5-(2-fluorophenyl)-2-pyrimidinyl]-3-oxospiro[5-azaisobenzofuran-1(3H),1′-cyclohexane]-4′-carboxamide; trans-N-[1-(3,5-difluorophenyl)-4-imidazolyl]-3-oxospiro[7-azaisobenzofuran-1(3H),1′-cyclohexane]-4′-carboxamide; trans-3-oxo-N-(1-phenyl-4-pyrazolyl)spiro[4-azaisobenzofuran-1(3H),1′-cyclohexane]4′-carboxamide; trans-N-[1-(2-fluorophenyl)-3-pyrazolyl]-3-oxospiro[6-azaisobenzofuran-1(3H),1′-cyclohexane]-4′-carboxamide; trans-3-oxo-N-(1-phenyl-3-pyrazolyl)spiro[6-azaisobenzofuran-1(3H),1′-cyclohexane]4′-carboxamide; trans-3-oxo-N-(2-phenyl-1,2,3-triazol-4-yl)spiro[6-azaisobenzofuran-1(3H),1′-cyclohexane]-4′-carboxamide; and pharmaceutically acceptable salts and esters thereof. All of the above recited U.S. patents and publications are incorporated herein by reference.

Other suitable pharmaceutical agents that may be administered in combination with the compounds of the present invention include agents designed to treat tobacco abuse (e.g., nicotine receptor partial agonists, bupropion hypochloride (also known under the tradename Zyban™) and nicotine replacement therapies), agents to treat erectile dysfunction (e.g., dopaminergic agents, such as apomorphine), ADD/ADHD agents (e.g., Ritalin™, Straftera™, Concerta™ and Adderall™), and agents to treat alcoholism, such as opioid antagonists (e.g., naltrexone (also known under the tradename ReVia™) and nalmefene), disulfiram (also known under the tradename Antabuse™), and acamprosate (also known under the tradename Campral™)). In addition, agents for reducing alcohol withdrawal symptoms may also be co-administered, such as benzodiazepines, beta-blockers, clonidine, carbamazepine, pregabalin, and gabapentin (Neurontin™). Treatment for alcoholism is preferably administered in combination with behavioral therapy including such components as motivational enhancement therapy, cognitive behavioral therapy, and referral to self-help groups, including Alcohol Anonymous (AA).

Other pharmaceutical agents that may be useful include antihypertensive agents; anti-inflammatory agents (e.g., COX-2 inhibitors); antidepressants (e.g., fluoxetine hydrochloride (Prozac™)); cognitive improvement agents (e.g., donepezil hydrochloride (Aircept™) and other acetylcholinesterase inhibitors); neuroprotective agents (e.g., memantine); antipsychotic medications (e.g., ziprasidone (Geodon™), risperidone (Risperdal™), and olanzapine (Zyprexa™)); insulin and insulin analogs (e.g., LysPro insulin); GLP-1 (7-37) (insulinotropin) and GLP-1 (7-36)—NH₂; sulfonylureas and analogs thereof: chlorpropamide, glibenclamide, tolbutamide, tolazamide, acetohexamide, Glypizide®, glimepiride, repaglinide, meglitinide; biguanides: metformin, phenformin, buformin; □2-antagonists and imidazolines: midaglizole, isaglidole, deriglidole, idazoxan, efaroxan, fluparoxan; other insulin secretagogues: linogliride, A-4166; glitazones: ciglitazone, Actos® (pioglitazone), englitazone, troglitazone, darglitazone, Avandia® (BRL49653); fatty acid oxidation inhibitors: clomoxir, etomoxir; □-glucosidase inhibitors: acarbose, miglitol, emiglitate, voglibose, MDL-25,637, camiglibose, MDL-73,945; □-agonists: BRL 35135, BRL 37344, RO 16-8714, ICI D7114, CL 316,243; phosphodiesterase inhibitors: L-386,398; lipid-lowering agents: benfluorex: fenfluramine; vanadate and vanadium complexes (e.g., Naglivan®) and peroxovanadium complexes; amylin antagonists; glucagon antagonists; gluconeogenesis inhibitors; somatostatin analogs; antilipolytic agents: nicotinic acid, acipimox, WAG 994, pramlintide (Symlin□), AC 2993, nateglinide, aidose reductase inhibitors (e.g., zopolrestat), glycogen phosphorylase inhibitors, sorbitol dehydrogenase inhibitors, sodium-hydrogen exchanger type 1 (NHE-1) inhibitors and/or cholesterol biosynthesis inhibitors or cholesterol absorption inhibitors, especially a HMG-CoA reductase inhibitor (e.g., atorvastatin or the hemicalcium salt thereof), or a HMG-CoA synthase inhibitor, or a HMG-CoA reductase or synthase gene expression inhibitor, a CETP inhibitor, a bile acid sequesterant, a fibrate, an ACAT inhibitor, a squalene synthetase inhibitor, an anti-oxidant or niacin. The compounds of the present invention may also be administered in combination with a naturally occurring compound that acts to lower plasma cholesterol levels. Such naturally occurring compounds are commonly called nutraceuticals and include, for example, garlic extract, Hoodia plant extracts, and niacin.

A compound of the present invention or a combination of a compound of the present invention and at least one additional pharmaceutical agent (referred to herein as a “combination”) is administered to a subject in need of such treatment, preferably in the form of a pharmaceutical composition. In the combination aspect of the invention, the combination may be administered either separately or in the pharmaceutical composition comprising both. It is generally preferred that such administration be oral. However, if the subject being treated is unable to swallow, or oral administration is otherwise impaired or undesirable, parenteral or transdermal administration may be appropriate.

When a compound of the present invention and the additional pharmaceutical agent are administered together, such administration can be sequential in time or simultaneous with the simultaneous method being generally preferred. For sequential administration, a compound of the present invention and the additional pharmaceutical agent can be administered in any order and may be by the same or different methods of administration. It is generally preferred that such administration be oral. It is especially preferred that such administration be oral and simultaneous.

A compound of the present invention or combination is preferably administered in the form of a pharmaceutical composition. A typical formulation is prepared by mixing a compound of the present invention with a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug or aid in the manufacturing of the pharmaceutical product (i.e., medicament).

The formulations may be prepared using conventional dissolution and mixing procedures. For example, the bulk drug substance (the compound or stabilized form of the compound (e.g., complex with a cyclodextrin derivative or other known complexation agent)) is dissolved in a suitable solvent in the presence of one or more of the excipients described above. The compound is typically formulated into pharmaceutical dosage forms to provide an easily controllable dosage of the drug and to give the patient an elegant and easily handleable product. To enhance dissolution rates, it may be advantageous to disperse poorly water-soluble compounds in a suitable dispersant prior to formulating into a dosage form. For example, the water-insoluble or partially water-insoluble compound may be spray-dried in the presence of a solubilizing or dispersing agent. See, e.g., Takeuchi, Hirofumi, et al., J Pharm Pharmacol, 39, 769-773 (1987). Other techniques for improving bioavailability of poorly water-soluble compounds are described in Verreck, G., et al., “The Use of Three Different solid Dispersion Formulations-Melt Extrusion, Film-coated Beads, and a Glass Thermoplastic System-to Improve the Bioavailability of a Novel Microsomal Triglyceride transfer Protein Inhibitor,” J Pharm Sci, 93(5),1217-1228 (2004).

For oral administration the pharmaceutical composition is generally administered in discrete units. For example, typical dosage forms include tablets, dragees, capsules, granules, sachets and liquid solutions or suspensions where each contain a predetermined amount of the active ingredient(s) in the form of a powder or granules, or as a solution or a suspension in an aqueous liquid, a non-aqueous liquid, an oil-in-water emulsion or a water-in-oil liquid emulsion.

Compressed tablets may be prepared by compressing the active ingredient(s) in a free-flowing form such as a powder or granules with a binder, lubricant, inert diluent, surface active agent and/or dispersing agent.

Liquid dosage forms for oral administration include pharmaceutically acceptable emulsions, solutions, suspensions, syrups, and elixirs. In addition to the active ingredient(s), the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, and flavoring agents.

Suspensions, in addition to the active ingredients, may further comprise suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The compounds can be administered by any method which delivers the compounds preferentially to the desired tissue (e.g., brain, renal or intestinal tissues). These methods include oral routes, parenteral, transdermal patches, intraduodenal routes, transdermal, etc. Generally, the compounds are administered orally in single (e.g., once daily) or multiple doses. The amount and timing of compounds administered will, of course, be dependent on the subject being treated, on the severity of the affliction, on the manner of administration and on the judgment of the prescribing physician. Thus, because of patient to patient variability, the dosages given herein are a guideline and the physician may titrate doses of the drug to achieve the treatment that the physician considers appropriate for the patient. In considering the degree of treatment desired, the physician generally balances a variety of factors such as age of the patient, presence of preexisting disease, lifestyle, as well as presence of other diseases (e.g., cardiovascular disease).

For human use, the daily dose of the compound of the present invention is generally between about 1.0 mg to about 100 mg, preferably between about 1.0 mg to about 50 mg, more preferably between about 2.0 mg to about 40 mg, most preferably between about 5.0 mg to about 25 mg. For non-human use, those skilled in the art know how to adjust the dosage for the particular weight of the animal.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 (available from Varian Inc., Palo Alto, Calif.) at room temperature at 400 MHz for proton. Chemical shifts are expressed in parts per million (δ) relative to residual solvent as an internal reference. The peak shapes are denoted as follows: s, singlet; d, doublet; t, triplet; q, quartet; m, multiplet; bs, broad singlet; 2s, two singlets. Atmospheric pressure chemical ionization mass spectra (APCI) were obtained on a Fisons™ Platform II Spectrometer (carrier gas: acetonitrile: available from Micromass Ltd, Manchester, UK). Chemical ionization mass spectra (Cl) were obtained on a Hewlett-Packard™ 5989 instrument (ammonia ionization, PBMS: available from Hewlett-Packard Company, Palo Alto, Calif.). Electrospray ionization mass spectra (ES) were obtained on a Waters™ ZMD instrument (carrier gas: acetonitrile: available from Waters Corp., Milford, Mass.). Where the intensity of chlorine or bromine-containing ions are described, the expected intensity ratio was observed (approximately 3:1 for ³⁵Cl/³⁷Cl-containing ions and 1:1 for ⁷⁹Br/⁸¹Br-containing ions) and the intensity of only the lower mass ion is given. In some cases only representative ¹H NMR peaks are given. MS peaks are reported for all examples. Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker™ silica gel (40 μm; J. T. Baker, Phillipsburg, N.J.) or Silica Gel 50 (EM Sciences™, Gibbstown, N.J.) in glass columns or in Flash 40 Biotage™ columns (ISC, Inc., Shelton, Conn.) under low nitrogen pressure.

The starting material, 5-(4-Chloro-phenyl)-1-(2-chloro-phenyl)-4-hydroxy-1H-pyrazole-3-carboxylic acid ethyl ester, may be prepared as described in U.S. patent application Ser. No. 10/971,599 entitled “Bicyclic Pyrazolyl and Imidazolyl Compounds and Uses Thereof” filed on Oct. 22, 2004 and incorporated herein by reference.

The following acronyms have the corresponding definitions:

AIBN 2,2′-azobisisobutyronitrile

DMAP 4-dimethylaminopyridine

### Starting Materials

Ethyl 1-(2-chlorophenyl)-5-(4-chlorophenyl)-4-hydroxy-1H-pyrazole-3-carboxylate may be prepared as described in U.S. patent application Ser. No. 10/971,599 entitled “Bicyclic Pyrazolyl and Imidazolyl Compounds and Uses Thereof” filed on Oct. 22, 2004 (incorporated herein by reference) and reproduced below.

Step 1: Bromine (15 mL, 294 mmol) was added in one portion to a cooled (ice/water bath) stirred solution of 5-(4-chlorophenyl)-1-(2-chlorophenyl)-1H-pyrazole-3-carboxylic acid ethyl ester (26.6 g, 73.6 mmol) in acetic acid (300 mL). After 45 minutes, the reaction was concentrated in vacuo, the solids slurried in diethyl ether (100 mL), filtered and dried in vacuo to afford 4-bromo-5-(4-chlorophenyl)-1-(2-chlorophenyl)-1H-pyrazole-3-carboxylic acid ethyl ester as a light-yellow colored solid, 29.6 g.

Step 2: A solution of 4-bromo-5-(4-chlorophenyl)-1-(2-chlorophenyl)-1H-pyrazole-3-carboxylic acid ethyl ester (5.2 g, 11.9 mmol), tributylvinyltin (7.0 mL, 23.8 mmol) and tetrakistriphenylphosphine palladium (0.7 g, 0.6 mmol) in DMF (12 mL) was heated at 110° C. for 18 hours. The dark solution was cooled, partitioned between ethyl ether/water, the organic layer washed with brine, dried (Na₂SO₄) and concentrated in vacuo to afford a semi-solid. This semi-solid was stirred with cyclohexanes (35 mL) and filtered to afford 5-(4-Chloro-phenyl)-1-(2-chloro-phenyl)-4-vinyl-1H-pyrazole-3-carboxylic acid as a white solid, (3.0 g).

Step 3: A solution of 5-(4-chlorophenyl)-1-(2-chlorophenyl)-4-vinyl-1H-pyrazole-3-carboxylic acid ethyl ester (2.9 g, 7.5 mmol), osmium tetroxide (8 mg, 0.08 mmol) and N-methylmorpholine-N-oxide (1.1 g, 8.2 mmol) in dioxane (24 mL)/water (6 mL) was stirred at ambient temperature for 18 hours, then sodium periodate (16 g, 75 mmol) was added and stirring was continued for 3.5 hours. The thick slurry was diluted with ethyl acetate (100 mL), filtered and solids washed 2× with ethyl acetate. The combined filtrates were washed with water, brine, dried (Na₂SO₄) and concentrated in vacuo to afford a solid mass. The solids were slurried in hot hexanes (30 mL), cooled, filtered and dried in vacuo to afford 5-(4-chlorophenyl)-1-(2-chlorophenyl)₄-formyl-1H-pyrazole-3-carboxylic acid ethyl ester as a tan solid, 2.2 g.

Final step: To a stirred solution of 5-(4-chlorophenyl)-1-(2-chlorophenyl)-4-formyl-1H-pyrazole-3-carboxylic acid ethyl ester (2.2 g, 5.6 mmol) in dichloromethane (22 mL) was added m-chloroperbenzoic acid (2.9 g (50% purity), 8.4 mmol) and the resulting slurry was stirred for 6 hours. The mixture was diluted into ethyl ether, washed with half-saturated aqueous sodium bicarbonate, water, brine, dried (Na₂SO₄) and concentrated in vacuo to afford a yellow solid, 3.5 g. To a slurry of this material in methanol (20 mL), was added triethylamine (1 mL) to produce a solution. After 45 minutes, the reaction was concentrated in vacuo to afford a yellow solid. This material was purified by silica gel chromatograpy (Combiflash instrument, 120 g silica gel column, 5-25% gradient of ethyl acetate/hexanes to afford 5-(4-Chloro-phenyl)-1-(2-chloro-phenyl)-4-hydroxy-1H-pyrazole-3-carboxylic acid ethyl ester as a yellow solid, 1.5 g.

### Preparation of Key Intermediates

### Preparation of Intermediate 4-(3-tert-butoxycarbonyl-propoxy)-1-(2-chloro-phenyl)-5-(4-chloro-phenyl)-1H-pyrazole-3-carboxylic acid ethyl ester (I-1a)

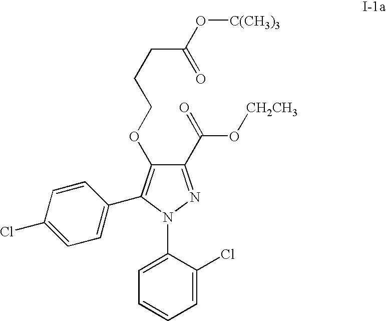

To a solution of ethyl 1-(2-chlorophenyl)-5-(4-chlorophenyl)-4-hydroxy-1H-pyrazole-3-carboxylate (20 g, 53 mmol) in DMF (100 mL) at 0° C. was added KOtBu (7.73 g, 69 mmol). The mixture was stirred at 0° C. for 45 minutes, then tert.-butyl 4-bromobutanoate (15.4 g, 69 mmol) was added at 0° C. The reaction mixture was allowed to warm to room temperature and stir overnight. The mixture was portioned between ether and saturated aqueous NH₄Cl. The organic solution was washed with water and saturated aqueous NaCl, dried over Na₂SO₄, and concentrated in vacuo. The residue was taken into hot cyclohexane. The mixture was allowed to stand for 72 hours. The solvent was decanted and the residue was washed with cold cyclohexane. The solid was then dried under high vacuum to yield 4-(3-tert.-butoxycarbonyl-propoxy)-1-(2-chloro-phenyl)-5-(4-chloro-phenyl)-1H-pyrazole-3-carboxylic acid ethyl ester (I-1a: 20.8 g).

¹H NMR (400 MHz, CHLOROFORM-D) □ ppm 1.4 (s, 9H), 1.4 (t, J=7.1 Hz, 3H), 1.9 (m, 2H), 2.3 (t, J=7.5 Hz, 2H), 4.0 (t, J=5.9 Hz, 2H), 4.4 (q, J=7.1 Hz, 2H), 7.2 (m, 2H), 7.2 (m, 2H), 7.4 (m, 3H), 7.5 (m, 1H); m/z=519.0 (M+1).

### Preparation of Intermediate 3-(4-chloro-phenyl)-2-(2-chloro-phenyl)-8-oxo-5,6,7,8-tetrahydro-2H-4-oxa-1,2-diaza-azulene-7-carboxylic acid tert-butyl ester (I-1b)

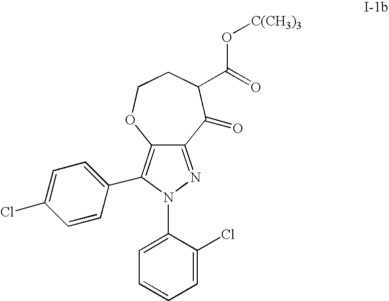

To a solution of KHMDS (112 mL of 0.5 M solution, 56 mmol) in THF (200 mL) at −78° C. was added dropwise a solution of 4-(3-tert.-butoxycarbonyl-propoxy)-1-(2-chloro-phenyl)-5-(4-chloro-phenyl)-1H-pyrazole-3-carboxylic acid ethyl ester (I-1a: 20.8 g, 40 mmol) in THF (200 mL). The mixture was stirred at −78° C. for 15 minutes and at 0° C. for 1 hour, then was quenched with saturated aqueous NH₄Cl. The mixture was partitioned between ether and water. The organic solution was dried over Na₂SO₄ and concentrated in vacuo. The residue was recrystallized from cyclohexane. The mixture was decanted and the solid was washed with cold cyclohexane to yield 3-(4-chloro-phenyl)-2-(2-chloro-phenyl)-8-oxo-5,6,7,8-tetrahydro-2H-4-oxa-1,2-diaza-azulene-7-carboxylic acid tert-butyl ester (I-1b) as a white powder (15.01 g). The mother liquor was concentrated and chromatographed (eluted with 10-30% ethyl acetate in hexane) to yield another 2g of the product.

¹H NMR (400 MHz, CHLOROFORM-D) □ ppm 1.5 (s, 9H), 2.9 (dd, J=4.8, 3.9 Hz, 2H), 4.3 (m, 2H), 7.2 (m, 2H), 7.2 (m, 2H), 7.4 (m, 3H), 7.5 (m, 1H), 13.2 (s, 1H); m/z=473.0 (M+1).

### Preparation of Intermediate 3-(4-chloro-phenyl)-2-(2-chloro-phenyl)-6,7-dihydro-2H,5H-4-oxa-1,2-diaza-azulen-8-one (I-1c)

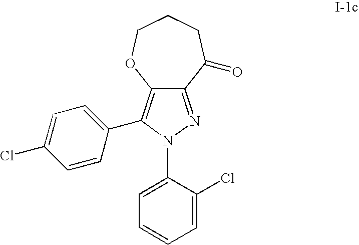

A solution of 3-(4-chloro-phenyl)-2-(2-chloro-phenyl)-8-oxo-5,6,7,8-tetrahydro-2H-4-oxa-1,2-diaza-azulene-7-carboxylic acid tert-butyl ester (I-1b: 15 g, 31.7 mmol) in 2:1 methylene chloride/trifluoroacetic acid (120 mL) was stirred at room temperature for 2 hours. Solvent was removed and the residue was taken up in 1:1 toluene/dioxane (200 mL). The solution was heated at reflux and then cooled to room temperature. The mixture was concentrated in vacuo and the residue was chromatographed on silica gel (eluted with a solvent gradient of 100% methylene chloride to 5% methanol/methylene chloride). The desired fractions were concentrated and the residue was triturated in cyclohexane to yield 3-(4-chloro-phenyl)-2-(2-chloro-phenyl)-6,7-dihydro-2H,5H-4-oxa-1,2-diaza-azulen-8-one (I-1c) as a pale yellow solid (10 g).

¹H NMR (400 MHz, CHLOROFORM-D) □ ppm 2.3 (m, 2H), 3.0 (dd, J=6.9, 5.6 Hz, 2H), 4.4 (m, 2H), 7.1 (m, 2H), 7.2 (m, 2H), 7.4 (m, 3H), 7.5 (m, 1H); m/z=373.0 (M+1).

### Preparation of Intermediate 4-Bromomethyl-1-(2-chloro-phenyl)-5-(4-chloro-phenyl)-1H-pyrazole-3-carboxylic acid ethyl ester (I-2a)

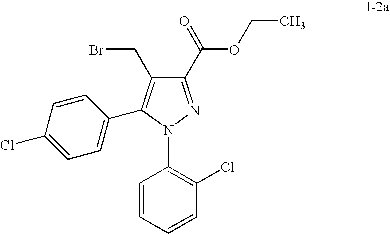

A mixture of 1-(2-chloro-phenyl)-5-(4-chloro-phenyl)-4-methyl-1H-pyrazole-3-carboxylic acid ethyl ester (see EP 656354 for preparation, 2.8 g, 7.46 mmol), N-bromosuccinimide (1.6 g, 8.95 mmol), AIBN (245 mg, 1.49 mmol) in CCl₄ (60 mL) was heated under reflux for 17 hours. The reaction mixture was cooled to room temperature, filtered to remove any solids, and concentrated under vacuum. The crude residue was purified via silica gel chromatography (Flash 40 system) using a solvent gradient of 10% EtOAc/hexanes to 20% EtOAc/hexanes to give the desired product (I-2a) as an amorphous solid (2.2 g, 64%): +APCI MS (M+1) 455.0.

### Preparation of Intermediate 1-(2-Chloro-phenyl)-5-(4-chloro-phenyl)-4-[(2-ethoxycarbonyl-ethylamino)-methyl]-1H-pyrazole-3-carboxylic acid ethyl ester (I-2b)

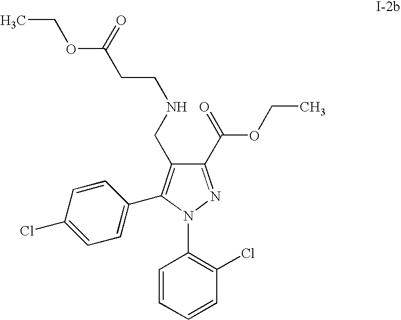

A mixture of 4-bromomethyl-1-(2-chloro-phenyl)-5-(4-chloro-phenyl)-1H-pyrazole-3-carboxylic acid ethyl ester (I-2a, 1208 mg, 2.66 mmol), □-alanine ethyl ester hydrochloride (2043 mg, 13.3 mmol), K₂CO₃ (1835 mg, 13.3 mmol) in DMF (20 mL) was stirred for 17 hours at room temperature. The reaction mixture was diluted with EtOAc and the organic solution was washed with H₂O and saturated aqueous NaCl, dried, and concentrated in vacuo to give (I-2b): +APCI MS (M+1) 490.2.

### Preparation of Intermediate 4-{[tert-Butoxycarbonyl-(2-ethoxycarbonyl-ethyl)-amino]-methyl}-1-(2-chloro-phenyl)-5-(4-chloro-phenyl)-1H-pyrazole-3-carboxylic acid ethyl ester (I-2c)

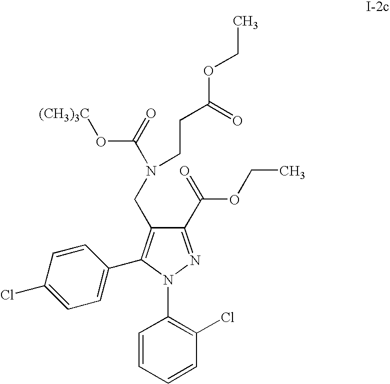

A solution of 1-(2-chloro-phenyl)-5-(4-chloro-phenyl)-4-[(2-ethoxycarbonyl-ethylamino)-methyl]-1H-pyrazole-3-carboxylic acid ethyl ester (I-2b) obtained from the previous step, di-tert.-butyl dicarbonate (870 mg, 3.99 mmol), DMAP (325 mg, 2.66 mmol) in CH₂Cl₂ was heated under reflux for 6 hours, cooled to room temperature, and concentrated in vacuo. The residue was diluted with CHCl₃, washed with 10% HCl (2×), saturated aqueous NaHCO₃ (1×), and saturated aqueous NaCl, dried, and concentrated under vacuum. The crude product was purified on SiO₂-gel using a solvent gradient of 15% ethyl acetate/hexanes to 100% EtOAc to give (I-2c) as an oil (520 mg): +APCI MS (M+1) 590.3.

### Preparation of Intermediate 2-(2-Chloro-Phenyl)-3-(4-chloro-Phenyl)-8-oxo-2,6,7,8-tetrahydro-4H-1,2,5-triaza-azulene-5,7-dicarboxylic acid 5-tert-butyl ester 7-ethyl ester (I-2d)

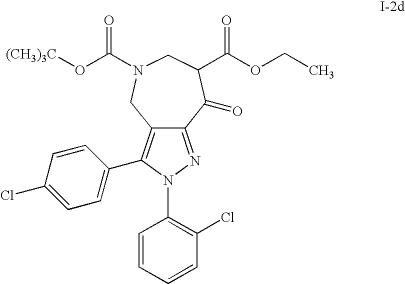

A solution of 4-{[tert-butoxycarbonyl-(2-ethoxycarbonyl-ethyl)-amino]-methyl}-1-(2-chloro-phenyl)-5-(4-chloro-phenyl)-1H-pyrazole-3-carboxylic acid ethyl ester (I-2c, 100 mg, 0.169 mmol) in anhydrous THF (2 mL) was added to a solution of 1 M potassium tert-butoxide/THF (2 mL) in THF (2 mL) at room temperature. The reaction mixture was stirred for 17 hours, diluted with EtOAc, and quenched with 1 M HCl. The organic layer was separated and washed with saturated aqueous NaCl, dried, and concentrated in vacuo. The crude residue was purified via a preparative chromatography plate using 30% ethyl acetate/hexanes to give the product (I-2d) as an oil (40 mg, 43%): +APCI MS (M+1) 544.2.

### Preparation of Intermediate 2-(2-Chloro-phenyl)-3-(4-chlorophenyl)-8-oxo-2,6,7,8-tetrahydro-4H-1,2,5-triaza-azulene-5-carboxylic acid tert-butyl ester (I-2e)

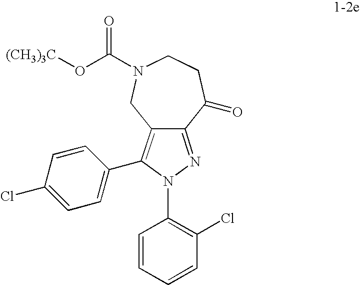

A mixture of 2-(2-chloro-phenyl)-3-(4-chloro-phenyl)-8-oxo-2,6,7,8-tetrahydro-4H-1,2,5-triaza-azulene-5,7-dicarboxylic acid 5-tert-butyl ester 7-ethyl ester (I-2d, 1000 mg, 1.84 mmol), NaCl (167 mg, 2.87 mmol) in H₂O/DMSO (0.1 mL/2.8 mL) was heated at 155° C. for 1.5 hours. The reaction mixture was cooled to room temperature and diluted with EtOAc. The organic solution was washed with H₂O, saturated aqueous NaCl, dried, and concentrated in vacuo. The crude residue was purified on a SiO₂-gel pad using a solvent gradient of 30% ethyl acetate/hexanes to 70% ethyl acetate/hexanes to give the product (I-2e) as an oil (540 mg, 62%): +APCI MS (M+1) 472.2.

### Preparation of Intermediate 1-(2-Chloro-phenyl)-4-formyl-5-(4-trifluoromethyl-phenyl)-1H-pyrazole-3-carboxylic acid ethyl ester (I-3a)

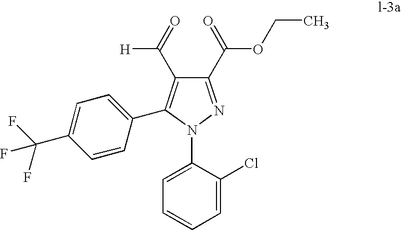

A mixture of ethyl 5-bromo-1-(2-chlorophenyl)-4-formyl-1H-pyrazole-3-carboxylate (see US Patent Publication No. 20040214855 for preparation, 1240 mg, 3.47 mmol), 4-(trifluoromethyl)-phenylboronic acid (990 mg, 5.21 mmol), solid cesium fluoride (1.55 g, 10.41 mmol) and bis(triphenylphosphine)palladium dichloride (210 mg, 0.30 mmol) in dimethoxyethane (50 mL) was heated in an 80° C. oil bath for 17 hours and cooled to room temperature. The supernatant was decanted from the dark insolubles. Additional 1,2-dimethoxyethane was added to the solid residues and decanted again. The combined organic solutions were diluted with ethyl acetate, washed with saturated aqueous NaHCO₃ and saturated aqueous NaCl, dried over sodium sulfate, and concentrated under vacuum to give (I-3a) as a thick residual oil: +APCI MS (M+1) 423.2.

### Preparation of Intermediate 1-(2-Chloro-phenyl)-4-[(2-ethoxycarbonyl-ethylamino)-methyl]-5-(4-trifluoromethyl-phenyl)-1H-pyrazole-3-carboxylic acid ethyl ester (I-3b)

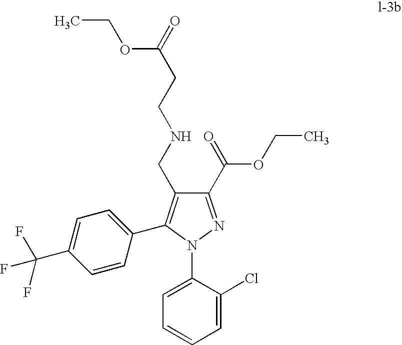

A solution of ethyl 1-(2-chlorophenyl)-4-formyl-5-[4-(trifluoromethyl)-phenyl]-1H-pyrazole-3-carboxylate obtained in the previous step (I-3a, 3.47 mmol) and ethyl-3-aminopropanoate hydrochloride (649 mg, 4.22 mmol) in 1,2-dichloroethane (50 mL) was stirred at room temperature for 0.17 hours. Solid sodium triacetoxyborohydride (1.42 g, 6.94 mmol) was added in one portion. The reaction mixture was stirred at room temperature under a nitrogen atmosphere for 4 hours, diluted with CH₂Cl₂, and quenched with 1 N NaOH. The organic layer was separated and the aqueous layer was extracted with CH₂Cl₂ The combined organic extracts were washed with saturated aqueous NaCl, dried, and concentrated to give (I-3b) as a residual oil (2200 mg): +APCI MS (M+1) 524.3.

### Preparation of Intermediate 4-{[tert-Butoxycarbonyl-(2-ethoxycarbonyl-ethyl)-amino]-methyl}-1-(2-chloro-Phenyl)-5-(4-trifluoromethyl-phenyl)-1H-pyrazole-3-carboxylic acid ethyl ester (I-3c)

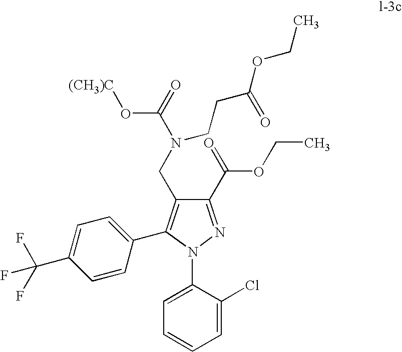

To a solution of 1-(2-chloro-phenyl)-4-[(2-ethoxycarbonyl-ethylamino)-methyl]-5-(4-trifluoromethyl-phenyl)-1H-pyrazole-3-carboxylic acid ethyl ester (I-3b, 3.47 mmol) in acetonitrile (75 mL) was added triethylamine (725 μl, 5.2 mmol) and di-tert.-butyl dicarbonate (1130 mg, 5.2 mmol). The reaction mixture was stirred at room temperature for 2 hours and quenched with saturated aqueous NaHCO₃. The organic layer was separated and the aqueous layer was extracted with ethyl acetate. The combined organic solutions were washed with saturated aqueous NaCl, dried, and concentrated in vacuo to give a residual amber oil which was dissolved in CH₂Cl₂ and adsorbed onto silica gel. The product was purified by chromatography through a Biotage 65 column, eluting with a solvent gradient of 15:5:0.5 CH₂Cl₂/hexanes/CH₃OH to 15:5:1.0 CH₂Cl₂/hexanes/CH₃OH to give 1.99 g (91% for three steps) of (I-3c) as a yellow viscous oil.

¹H NMR (400 MHz, CD₃OD) δ ppm 1.18-1.23 (t, 3H, J=7.1 Hz), 1.28 (s, 9H), 1.37-1.42 (t, 3H, J=7.1 Hz), 2.33-2.40 (t, 2H, J=71. Hz), 3.14-3.20 (t, 2H, J=7.1 Hz), 4.0-4.1 (t, 2H, J=7.1 Hz), 4.38-4.42 (t, 2H, J=7.1 Hz), 4.81 (s, 2H), 7.36-7.66 (m, 8H); +APCI MS (M+1) 624.4.

### Preparation of Intermediate 2-(2-Chloro-phenyl)-8-oxo-3-(4-trifluoromethyl-phenyl)-2,6,7,8-tetrahydro-4H-1,2,5-triaza-azulene-5,7-dicarboxylic acid 5-tert-butyl ester 7-ethyl ester (I-3d)

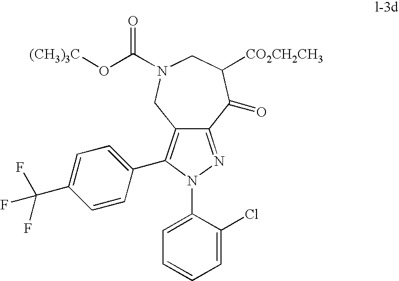

A solution of 4-{[tert-butoxycarbonyl-(2-ethoxycarbonyl-ethyl)-amino]-methyl}-1-(2-chloro-phenyl)-5-(4-trifluoromethyl-phenyl)-1H-pyrazole-3-carboxylic acid ethyl ester (I-3c,1980 mg, 3.17 mmol) in toluene (150 mL) was heated under reflux in a round bottom flask with a sidearm condenser attached. The solvent was removed via distillation. The residue was diluted with THF (150 mL) and concentrated via distillation to a volume of −50 mL. The solution was cooled to room temperature and diluted with additional THF (100 mL). Potassium hexamethyldisilazide (14 mL of 0.5 M in toluene, 7.0 mmol) was added at room temperature via syringe over a 7-8 minute period. The reaction mixture was stirred at room temperature for 2 hours, quenched with aqueous citric acid (50 mL of 0.5 M solution), diluted with ethyl acetate, and washed with saturated aqueous NaCl. The organic layer was separated and the aqueous layer was extracted with EtOAc. The combined organic extracts were washed with saturated aqueous NaCl, dried, and concentrated to give 2.2 g of (I-3d) as a yellow glass foam (theoretical yield=1.83 g): +APCI MS (M+1) 578.4.

### Preparation of Intermediate 2-(2-Chloro-phenyl)-8-oxo-3-(4-trifluoromethyl-phenyl)-2,6,7,8-tetrahydro-4H-1,2,5-triaza-azulene-5-carboxylic acid tert-butyl ester (I-3e)

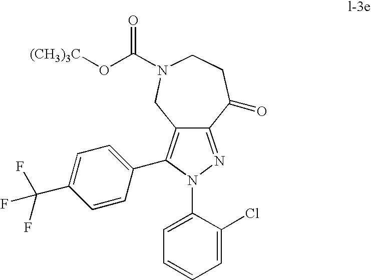

To a solution of 2-(2-chloro-phenyl)-8-oxo-3-(4-trifluoromethyl-phenyl)-2,6,7,8-tetrahydro-4H-1,2,5-triaza-azulene-5,7-dicarboxylic acid 5-tert-butyl ester 7-ethyl ester (I-3d, 2300 mg, 3.1 mmol) in DMSO (10 mL) at room temperature was added sodium chloride (362 mg, 6.2 mmol) and H₂O (279 μL, 15.5 mmol). The reaction mixture was immersed in a preheated 155° C. oil bath and heated for 1.75 hours. The dark mixture was cooled to room temperature, diluted with saturated aqueous NaCl, and extracted with EtOAc (3×). The combined extracts were washed with saturated aqueous NaCl, dried, and concentrated to give 1.6 g of crude (I-3e) as a dark, glassy residue. The residue was purified by radial chromatography (4 mm silica gel plate; eluent—50% EtOAc/hexanes) to give 476 mg yellow oil (30% yield for two steps). NMR shows evidence of rotamers of the desired product.

¹H NMR (400 MHz, CD₃Cl) δ ppm 1.26 (s, 3.1H), 1.43 (s, 5.9H), 2.95-3.10 (m, 2H), 3.7-3.8 (m, 2H), 4.60 (s, 0.4H), 4.68 (s, 0.6H), 7.25-7.60 (m, 8H); +APCI MS (M+1) 506.2.

### Example 1

### Preparation of (R,S)-3-(4-Chloro-phenyl)-2-(2-chloro-phenyl)-5,6,7,8-tetrahydro-2H-4-oxa-1,2-diaza-azulen-8-ylamine (1A)hydrochloride (1A-1)

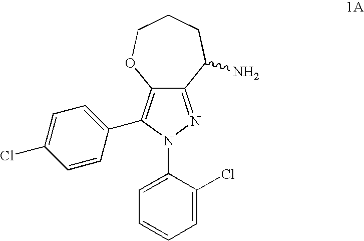

To a stirred solution of 3-(4-chloro-phenyl)-2-(2-chloro-phenyl)-6,7-dihydro-2H,5H-4-oxa-1,2-diaza-azulen-8-one (I-1c: 10.0 g, 26.8 mmol) and ammonium acetate (20.6 g, 268 mmol) in 2:1 methanol/dichloromethane (135 mL) was added sodium cyanoborohydride (1.68 g, 26.8 mmol). After 4 hours at ambient temperature, 6N sodium hydroxide (10 mL) was added and stirring was continued for 15 minutes. The reaction mixture was concentrated in vacuo, diluted into ethyl acetate, washed with saturated saturated aqueous NaCl, dried over Na₂SO₄ and concentrated in vacuo. The resulting yellow foam was dissolved in 5% ethyl acetate/diethyl ether (200 mL) and then treated with 4 M hydrochloric acid in dioxane (10 mL). The resulting solid was filtered, washed with diethyl ether, and dried in vacuo to afford the hydrochloride salt of the title compound (1A) as a pale yellow solid (6.2 g).

¹H NMR (400 MHz, Methanol-D₄) □ ppm 1.92 (q, 1H), 2.08-2.34 (m, 3H), 3.81 (t, 1H), 4.31 (dd, 1H), 4.56 (dd, 1H), 7.15 (d, 2H), 7.25 (d, 2H), 7.43-7.53 (m, 4H); m/z=374.0 (M+1).

Separation of Enantiomers:

The racemic mixture of 3-(4-chloro-phenyl)-2-(2-chloro-phenyl)-5,6,7,8-tetrahydro-2H-4-oxa-1,2-diaza-azulen-8-ylamine (1A: 4.0 g) was purified by chiral phase HPLC (Chiralpak AD column, 10 cm×50 cm, flow rate=275 mL/min, mobile phase=heptane:ethanol 92:8, with 0.1% diethylamine, 500 mg injections (dissolved in 2 mL of 1:1 methanol:dichloromethane) to afford “enantiomer 1” (enantiomeric excess of 98%) which has an approximate retention time of 39 minutes and “enantiomer 2” (enantiomeric excess of 97%) which has an approximate retention time of 49 minutes. The enantiomers were concentrated in vacuo and converted to their respective hydrochloride salts as described above for the racemate.

¹H NMR and mass spectral data are consistent with those reported for the racemic amine hydrochloride salt.

### Preparation of [3-(4-Chloro-phenyl)-2-(2-chloro-phenyl)-5,6,7,8-tetrahydro-2H-4-oxa-1,2-diaza-azulen-8-yl]-(2,2,2-trifluoro-ethyl)-amine (1B)

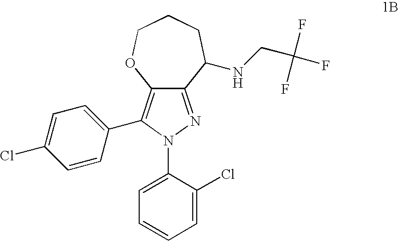

A mixture of 3-(4-chloro-phenyl)-2-(2-chloro-phenyl)-6,7-dihydro-2H,5H-4-oxa-1,2-diaza-azulen-8-one (I-1c: 1 g, 2.7 mmol), 2,2,2-trifluoroethylamine (316 □L, 4.0 mmol), sodium triacetoxyborohydride (850 mg, 4.0 mmol) and acetic acid (184 □L, 3.2 mmol) in 1,2-dichloroethane (10 mL) was stirred at ambient temperature for 18 hours. The reaction was partitioned between ethyl acetate and saturated aqueous sodium bicarbonate. The organic solution was dried over Na₂SO₄ and concentrated in vacuo. The resulting oil was loaded on a silica gel samplet and chromatographed on a Combiflash™ apparatus (12 g silica gel cartridge, 10-30% ethyl acetate:hexanes gradient) to afford the title compound (1B) as a white powder (995 mg);

¹H NMR (400 MHz, Chloroform-D) □ppm 1.94-2.10 (m, 3H), 2.27-2.38 (m, 1H), 3.20-3.40 (m, 2H), 3.94 (t, 1H), 4.05-4.20 (m, 2H), 7.10-7.45 (m, 8H); m/z=456.1 (M+1).

The compounds listed in Table 1 were prepared utilizing procedures analogous to those described above for the preparation of Compounds 1A, 1A-1 and 1B using the appropriate starting materials.

TABLE 1

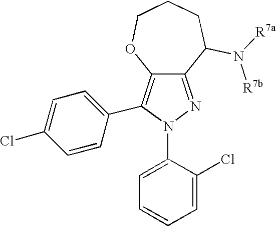

| | | | Mass Spec |
|---|---|---|---|
| Example No. | R^{7a} | R^{7b} | m/z (M + 1) |
| 1C | H | —CH₃ | 388.3 |
| 1D | H | —CH₂C═CH | 414.1 |

### Example 2

### Preparation of (R,S)-N-[3-(4-Chloro-phenyl)-2-(2-chloro-phenyl)-5,6,7,8-tetrahydro-2H-4-oxa-1,2-diaza-azulen-8-yl]-acetamide (2A);

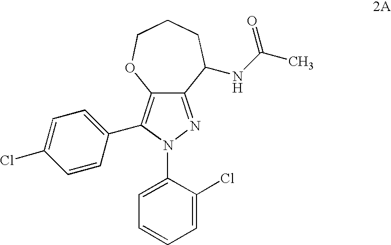

To a stirred, cooled (ice/water bath) solution of (R,S)-3-(4-chloro-phenyl)-2-(2-chloro-phenyl)-5,6,7,8-tetrahydro-2H-4-oxa-1,2-diaza-azulen-8-ylamine hydrochloride (1A-1: 33 mg, 0.09 mmol) and triethylamine (15 □L, 0.1 mmol) was added acetyl chloride (7 □L, 0.1 mmol). The reaction mixture was allowed to warm to ambient temperature and stir for 3 hours. The mixture was directly loaded onto a silica gel samplet and chromatographed on a Combiflash™ apparatus (4 g, eluting with a solvent gradient of 25% ethyl acetate/hexanes to 100% EtOAc) to afford the title compound (2A) as a yellow solid (10 mg).

¹H NMR (400 MHz, CHLOROFORM-D) □ ppm 1.59-1.69 (m, 2H), 2.05 (s, 3H), 2.06-2.23 (m, 1H), 2.44-2.49 (m, 1H), 3.73 (t, 1H), 4.24-4.29 (m, 1H), 5.08-5.13 (m, 1H), 6.63 (d, 1H), 7.13 (d, 2H), 7.21 (d, 2H), 7.34-7.43 (m, 4H); m/z=416.0 (M+1).

The compounds listed in Table 2 were prepared utilizing procedures analogous to those described above for the preparation of Compound 2A using the appropriate starting materials.

TABLE 2

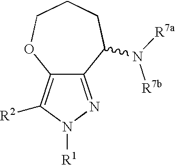

| Ex. | | | | | Mass Spec |
|---|---|---|---|---|---|
| No. | R¹ | R² | R^{7a} | R^{7b} | m/z (M + 1) |
| 2B | 2-chloro- phenyl | 4-chloro- phenyl | H | 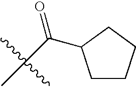 | 470.1 |
| 2C | 2-chloro- | 4-chloro- | H | —C(O)—CH(CH₃)₂ | 444.1 |
| | phenyl | phenyl | | | |
| 2D | 2-chloro- | 4-chloro- | H | —C(O)—CH₂CH₃ | 430.1 |
| | phenyl | phenyl | | | |
| 2E | 2-chloro- phenyl | 4-chloro- phenyl | 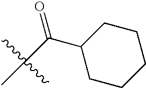 | 484.1 | |
| 2F | 2-chloro- | 4-chloro- | H | —C(O)—(CH₂)₃—Cl | 478.1 |
| | phenyl | phenyl | | | |
| 2G | 2-chloro- | 4-chloro- | —CH₂CF₃ | —C(O)—CH₂CH₃ | 512.1 |
| | phenyl | phenyl | | | |
| 2H | 2-chloro- phenyl | 4-chloro- phenyl | —CH₃ | 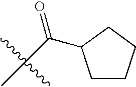 | 484.1 |
| 2I | 2-chloro- | 4-chloro- | —CH₃ | —C(O)—CH(CH₃)₂ | 458.1 |
| | phenyl | phenyl | | | |
| 2K | 2-chloro- | 4-chloro- | —CH₃ | —C(O)—CH₃ | 430.0 |
| | phenyl | phenyl | | | |
| 2L | 2-chloro- | 4-chloro- | —CH₂CF₃ | —C(O)—CH(CH₃)₂ | 526.1 |
| | phenyl | phenyl | | | |
| 2M | 2-chloro- phenyl | 4-chloro- phenyl | —CH₂CF₃ | 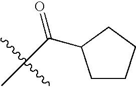 | 552.1 |
| 2N | 2-chloro- phenyl | 4-chloro- phenyl | H | 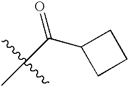 | 456.1 |
| 2O | 2-chloro- | 4-chloro- | H | —C(O)—CH₂—O—CH₂CH₃ | 460.1 |
| | phenyl | phenyl | | | |
| 2P | 2-cyano- | 4-chloro- | H | —C(O)—CH₃ | 407.0 |
| | phenyl | phenyl | | | |
| 2Q | 2-chloro- phenyl | 4-chloro- phenyl | H | 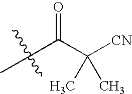 | 469.1 |
| 2R | 2-chloro- | 4-chloro- | H | —C(O)—CH₂—O—(CH₂)₃CH₃ | 488.1 |
| | phenyl | phenyl | | | |
| 2S | 2-chloro- phenyl | 4-chloro- phenyl | H | 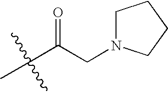 | 485.1 |
| 2T | 2-chloro- phenyl | 4-chloro- phenyl | H | 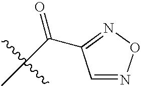 | 470.1 |
| 2U | 2-cyano- | 4-chloro- | H | —C(O)—CH(CH₃)₂ | 421.1 |
| | phenyl | phenyl | | | |
| 2V | 2-cyano- | 4-chloro- | H | —C(O)—CH(CH₃)₂ | 435.1 |
| | phenyl | phenyl | | | |
| 2W | 2-cyano- | 4-chloro- | H | —C(O)—CH₂CH₃ | 489.1 |
| | phenyl | phenyl | | | |
| 2X | 2-chloro- | 4-chloro- | H | —C(O)—CH₂—O—CH₃ | 446.0 |
| | phenyl | phenyl | | | |
| 2Y | 2-chloro- | 4-chloro- | H | —C(O)—CH₂—O—C(CH₃)₃ | 488.1 |
| | phenyl | phenyl | | | |
| 2Z | 2-chloro- | 4-chloro- | H | —C(O)—(CH₂)₂—O—CH₂CH₃ | 474.1 |
| | phenyl | phenyl | | | |
| 2AA | 2-chloro- | 4-chloro- | H | —C(O)—CH₂—O—CH₂CH₂— | 490.1 |
| | phenyl | phenyl | | OCH₃ | |
| 2AB | 2-chloro- | 4-chloro- | H | —C(O)—(CH₂)₃—OCH₃ | 474.0 |
| | phenyl | phenyl | | | |
| 2AC | 2-chloro- phenyl | 4-chloro- phenyl | H | 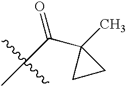 | 456.0 |
| 2AD | 2-chloro- | 4-chloro- | H | —C(O)—C(CH₃)₃ | 458.0 |
| | phenyl | phenyl | | | |
| 2AE | 2-chloro- phenyl | 4-chloro- phenyl | H | 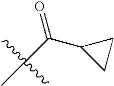 | 442.0 |
| 2AF | 2-chloro- | 4-chloro- | H | —C(O)—CH₂CH(CH₃)₂ | 458.0 |
| | phenyl | phenyl | | | |
| 2AG | 2-chloro- phenyl | 4-chloro- phenyl | H | 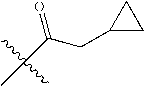 | 456.0 |
| 2AH | 2-chloro- | 4-chloro- | H | —C(O)—(CH₂)₃CH₃ | 458.0 |
| | phenyl | phenyl | | | |
| 2AI-1 | 2-chloro- | 4-chloro- | H | —C(O)—CH₂CH₃ | 430.0 |
| (R) | phenyl | phenyl | | | |
| 2AI-2 | 2-chloro- | 4-chloro- | H | —C(O)—CH₂CH₃ | 430.0 |
| (S) | phenyl | phenyl | | | |
| 2AJ-1 | 2-chloro- | 4-chloro- | H | —C(O)—CH(CH₃)₂ | 444.0 |
| (R) | phenyl | phenyl | | | |
| 2AJ-2 | 2-chloro- | 4-chloro- | H | —C(O)—CH(CH₃)₂ | 444.0 |
| (S) | phenyl | phenyl | | | |
| 2AK | 2-chloro- | 4-cyano- | H | —C(O)—CH₃ | 407.0 |
| | phenyl | phenyl | | | |
| 2AL | 2-chloro- | 4-cyano- | H | —C(O)—CH₂CH₃ | 421.0 |
| | phenyl | phenyl | | | |
| 2AM | 2-chloro- | 4-cyano- | H | —C(O)—CH(CH₃)₂ | 435.0 |
| | phenyl | phenyl | | | |
| 2AN | 2-chloro- | 4-cyano- | H | —C(O)—(CH₂)₂CF₃ | 489.0 |
| | phenyl | phenyl | | | |
| 2AO | 2-chloro- phenyl | 4-chloro- phenyl | H | 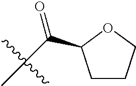 | 472.0 |
| 2AP | 2-chloro- phenyl | 4-chloro- phenyl | H | 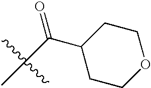 | 486.0 |
| 2AQ | 2-chloro- | 4-chloro- | H | —C(O)—CH₂C(CH₃)₃ | 472.0 |
| | phenyl | phenyl | | | |
| 2AR | 2-chloro- phenyl | 4-chloro- phenyl | H | 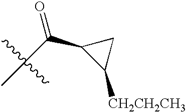 | 484.0 |
| 2AS | 2-chloro- phenyl | 4-chloro- phenyl | H | 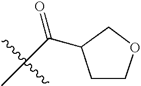 | 472.0 |
| 2AT | 2-chloro- phenyl | 4-chloro- phenyl | H | 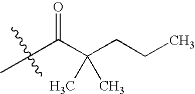 | 486.1 |
| 2AU | 2-chloro- | 4-chloro- | H | —C(O)—CH₂SO₂CH₃ | 494.0 |
| | phenyl | phenyl | | | |
| 2AV | 2-chloro- phenyl | 4-chloro- phenyl | H | 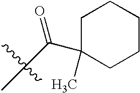 | 498.1 |
| 2AW | 2-chloro- phenyl | 4-chloro- phenyl | H | 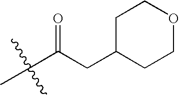 | 500.0 |
| 2AX | 2-chloro- phenyl | 4-chloro- phenyl | H | 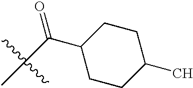 | 498.1 |
| 2AY | 2-chloro- phenyl | 4-chloro- phenyl | H | 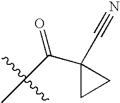 | 467.0 |
| 2AZ | 2-chloro- phenyl | 4-chloro- phenyl | H | 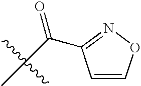 | 468.9 |
| 2BA | 2-chloro- phenyl | 4-chloro- phenyl | H | 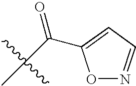 | 469.0 |
| 2BB | 2-chloro- phenyl | 4-chloro- phenyl | H | 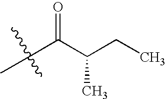 | 458.0 |
| 2BC | 2-chloro- | 4-chloro- | H | —C(O)—(CH₂)₃—CN | 469.0 |
| | phenyl | phenyl | | | |
| 2BD | 2-chloro- | 4-chloro- | H | —C(O)—(CH₂)₂—N(CH₃)₂ | 473.0 |
| | phenyl | phenyl | | | |
| 2BE | 2-chloro- | 4-chloro- | H | —C(O)—CH₂—O(CH₂)₂CH₃ | 474.0 |
| | phenyl | phenyl | | | |
| 2BF | 2-chloro- phenyl | 4-chloro- phenyl | H | 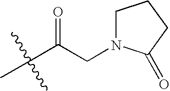 | 499.0 |
| 2BG | 2-chloro- | 4-chloro- | H | —C(O)—(CH₂)₂CF₃ | 498.0 |
| | phenyl | phenyl | | | |

### Example 3

### Preparation of 1-[3-(4-Chloro-phenyl)-2-(2-chloro-phenyl)-5,6,7,8-tetrahydro-2H-4-oxa-1,2-diaza-azulen-8-(R,S)-yl]-pyrrolidin-2-one (3A)

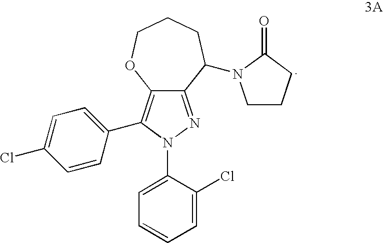

To a stirred solution of 4-chloro-N-[3-(4-chloro-phenyl)-2-(2-chloro-phenyl)-5,6,7,8-tetrahydro-2H-oxa-1,2-diaza-azulen-8-yl]-butyramide (2F: 165 mg, 0.44 mmol) in THF (0.6 mL) was added potassium tert.-butoxide (23 mg, 0.2 mmol). After 2 hours, the reaction was concentrated, loaded on a silica gel samplet, and chromatographed on a Combiflash™ apparatus (4 g column, 50-100% ethyl acetate:hexanes gradient) to afford the title compound (3A) as a white solid (50 mg).

¹H NMR (400 MHz, CHLOROFORM-D) □ ppm 1.96-2.21 (m, 6H), 2.42-2.47 (m, 2H), 3.43-3.48 (m, 1H), 3.62-3.68 (m, 1H), 3.90-3.95 (m, 1H), 4.12-4.17 (m, 1H), 5.45 (dd, 1H), 7.10 (d, 2H), 7.20-7.42 (m, 6H); m/z=442.1 (M+1).

### Example 4

### Preparation of [3-(4-Chloro-phenyl)-2-(2-chloro-phenyl)-5,6,7,8-tetrahydro-2H-4-oxa-1,2-diaza-azulen-8-(R,S)-yl]-carbamic acid isopropyl ester (4A)

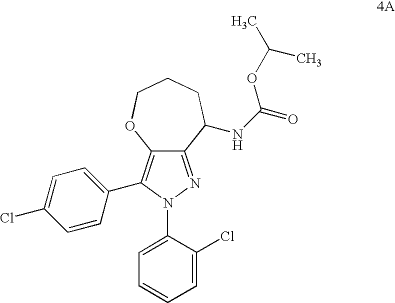

To a stirred solution of 3-(4-chloro-phenyl)-2-(2-chloro-phenyl)-5,6,7,8-tetrahydro-2H-4-oxa-1,2-diaza-azulen-8-ylamine, hydrochloride (1A-1: 30 mg, 0.07 mmol) and triethylamine (20 □L, 0.15 mmol) in dichloromethane (0.2 mL) was added isopropyl chloroformate (0.11 mL, 0.11 mmol). After 18 hours, the reaction mixture was loaded onto a silica gel samplet cartridge and chromatographed on a Combiflash™ apparatus (4 g silica gel column, eluting with a solvent gradient of 1:1 dichloromethane/hexanes to 10% methanol in 1:1 dichloromethane/hexanes) to afford the title compound (4A) as a white, waxy solid (14 mg).

¹H NMR (400 MHz, CHLOROFORM-D) □ ppm 1.09-1.25 (m, 3H), 1.60-1.80 (2H), 2.40-2.50 (m, 1H), 3.65-3.80 (m, 1H), 4.18-4.25 (m, 1H), 4.83-5.00 (m, 2H), 5.78 (br s, 1H), 7.08-7.43 (m, 8H); m/z=460.1 (M+1).

The compounds listed in Table 3 were prepared utilizing procedures analogous to those described above for the preparation of Compound 4A using the appropriate starting materials.

TABLE 3

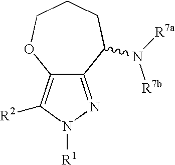

| | | | | | Mass Spec |
|---|---|---|---|---|---|
| Ex. | | | | | m/z |
| No. | R¹ | R² | R^{7a} | R^{7b} | (M + 1) |
| 4B | 2-chloro- | 4-chloro- | H | —C(O)—OCH₃ | 432.0 |
| | phenyl | phenyl | | | |
| 4C | 2-cyano- | 4-chloro- | H | —C(O)—OC(CH₃)₃ | 465.1 |
| | phenyl | phenyl | | | |
| 4D | 2-chloro- | 4-cyano- | H | —C(O)—OCH₂CH₃ | 437.0 |
| | phenyl | phenyl | | | |
| 4E | 2-chloro- | 4-cyano- | H | —C(O)—OCH(CH₃)₂ | 451.0 |
| | phenyl | phenyl | | | |
| 4F | 2-chloro- | 4-cyano- | H | —C(O)—O(CH₂)₂CH₃ | 451.0 |
| | phenyl | phenyl | | | |
| 4G | 2-chloro- | 4-chloro- | H | —C(O)—O(CH₂)₂OCH₃ | 476.0 |
| | phenyl | phenyl | | | |
| 4H | 2-chloro- | 4-chloro- | H | —C(O)—O(CH₂)₂CH₃ | 460.0 |
| | phenyl | phenyl | | | |
| 4I | 2-chloro- | 4-chloro- | H | —C(O)—OCH₂CH₃ | 446.0 |
| | phenyl | phenyl | | | |
| 4J | 2-chloro- | 4-chloro- | H | —C(O)—OC(CH₃)₃ | 474.0 |
| | phenyl | phenyl | | | |

### Example 5

### Preparation of 1-[3-(4-Chloro-phenyl)-2-(2-chloro-phenyl)-5,6,7,8-tetrahydro-2H-4-oxa-1,2-diaza-azulen-8-yl]-3-ethyl-urea (5A)

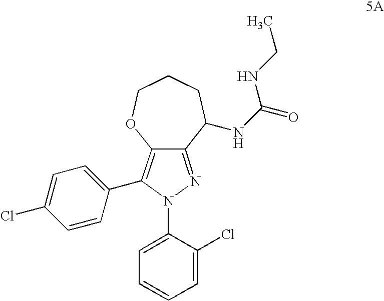

A solution of 3-(4-chloro-phenyl)-2-(2-chloro-phenyl)-5,6,7,8-tetrahydro-2H-4-oxa-1,2-diaza-azulen-8-ylamine, hydrochloride (1A-1: 30 mg, 0.07), triethylamine (0.02 mL, 0.15 mmol) and ethyl isocyanate (9 □L, 0.11 mmol) in dichloromethane (0.3 mL) were stirred at ambient temperature for 20 hours. The reaction solution mixture was loaded onto a silica gel samplet cartridge and chromatographed on a Combiflash™ apparatus (4 g silica gel column, eluting with a solvent gradient of 1:1 dichloromethane:hexanes to 10% methanol in 1:1 dichloromethane:hexanes) to afford the title compound (5A) as a waxy solid (10 mg).

¹H NMR (400 MHz, CHLOROFORM-D) □ ppm 1.07 (t, 3H), 1.70-1.80 (m, 1H), 2.03-2.16 (m, 2H), 2.33-2.41 (m, 1H), 3.11 (q, 2H), 3.78-4.08 (m, 1H), 4.92 (d, 1H), 7.08-7.42 (m, 8H); m/z=445.1 (M+1).

The compounds listed in Table 4 were prepared utilizing procedures analogous to those described above for the preparation of Compound 5A using the appropriate starting materials.

TABLE 4

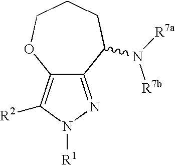

| No. | | | | | Mass Spec |
|---|---|---|---|---|---|
| Ex. | R¹ | R² | R^{7a} | R^{7b} | m/z (M + 1) |
| 5B | 2-chloro- | 4-chloro- | H | —C(O)—N(CH₂CH₃)₂ | 473.1 |
| | phenyl | phenyl | | | |
| 5C | 2-chloro- | 4-cyano- | H | —C(O)—NH(CH₂CH₃) | 436.0 |
| | phenyl | phenyl | | | |
| 5D | 2-chloro- | 4-cyano- | H | —C(O)—NH(CH₂CH₂CH₃) | 450.0 |
| | phenyl | phenyl | | | |
| 5E | 2-chloro- | 4-cyano- | H | —C(O)—NH(CH(CH₃)₂) | 494.0 |
| | phenyl | phenyl | | | |
| 5F | 2-chloro- phenyl | 4-cyano- phenyl | H | 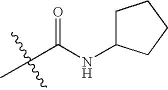 | 476.0 |
| 5G | 2-chloro- | 4-cyano- | H | —C(O)—NH(C(CH₃)₃) | 464.1 |
| | phenyl | phenyl | | | |
| 5H | 2-chloro- phenyl | 4-chloro- phenyl | H | 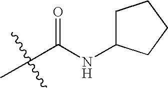 | 485.1 |
| 5I | 2-chloro- | 4-chloro- | H | —C(O)—NH(C(CH₃)₃) | 473.0 |
| | phenyl | phenyl | | | |
| 5J | 2-chloro- | 4-chloro- | H | —C(O)—NH(CH(CH₃)₂) | 459.0 |
| | phenyl | phenyl | | | |
| 5K | 2-chloro- | 4-chloro- | H | —C(O)—NH(CH₂CH₂CH₃) | 459.0 |
| | phenyl | phenyl | | | |
| 5L | 2-chloro- phenyl | 4-chloro- phenyl | H | 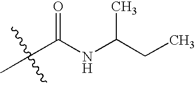 | 473.0 |
| 5M | 2-chloro- phenyl | 4-chloro- phenyl | H | 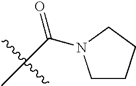 | 471.0 |

### Example 6

### Preparation of 8-Amino-2-(2-chloro-phenyl)-3-(4-trifluoromethyl-phenyl)-2,6,7,8-tetrahydro-4H-1,2,5-triaza-azulene-5-carboxylic acid tert-butyl ester (6A)

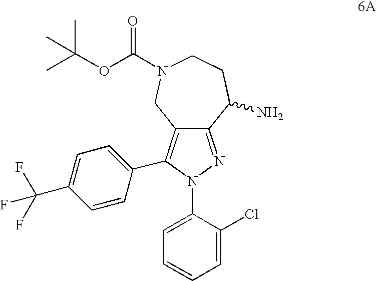

To a solution of 2-(2-chlorophenyl)-N-t-butoxycarbonyl-3-[4-(trifluoromethyl)phenyl]-4,5,6,7-tetrahydropyrazolo[4,3-c]azepin-8(2H)-one (I-3e, 69 mg, 0.13 mmol) in anhydrous methanol (1.0 mL) was added ammonium acetate (102 mg, 1.30 mmol) and six pellets of oven-dried 3A molecular sieves. The reaction mixture was stirred under a nitrogen atmosphere for 0.17 hours. Solid sodium cyanoborohydride (8.0 mg, 0.13 mmol) was added and the reaction mixture was reflushed with nitrogen, stirred at room temperature for 5 hours, quenched with 1 M K₂CO₃, and extracted with EtOAc (3×). The combined extracts were washed with saturated aqueous NaCl, dried, and concentrated to give 57 mg of (6A) as a yellow, tacky solid. Radial chromatographic purification (1 mm plate; 10:1 EtOAc/MeOH as eluent) yielded 22 mg product as an amorphous glass. NMR suggests a mix of rotamers.

¹H NMR (400 MHz, CD₃Cl) δ ppm 1.08 (s, 4.5H), 1.43 (s, 4.5H), 1.8-2.0 (m, 1H), 2.1-2.2 (m, 1H), 2.65 (brs, 2H), 3.4-3.6 (m, 1H), 4.04.15 (m, 1H), 4.24.3 (m, 1H), 4.3-4.4 (m, 1H), 4.54.6 (m, 1H), 7.2-7.6 (m, 8H); +APCI MS (M+1) 507.2.

### Example 7

### Preparation of N-[2-(2-Chloro-phenyl)-3-(4-trifluoromethyl-phenyl)-2,4,5,6,7,8-hexahydro-1,2,5-triaza-azulen-8-yl]-isobutyramide Hydrochloride (7A)

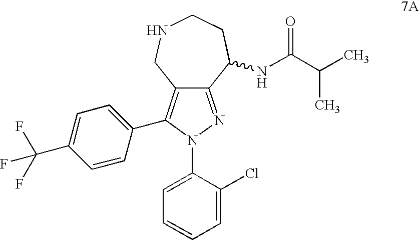

Step 1: To a solution of 8-amino-2-(2-chloro-phenyl)-3-(4-trifluoromethyl-phenyl)-2,6,7,8-tetrahydro-4H-1,2,5-triaza-azulene-5-carboxylic acid tert-butyl ester (6A, 7.4 mg, 14.6 μmol) in CH₂Cl₂ (100 μL) was added triethylamine (2.2 μL, 16.1 μmol) and isobutyryl chloride (1.7 μL, 16.1 μmol). The reaction mixture was stired for 2 hours at room temperature, quenched with 1 M aqueous K₂CO₃, and extracted with EtOAc (3×). The combined extracts were washed with saturated aqueous NaCl, dried, and concentrated under vacuum. The crude residue was purified by radial chromatography (1 mm silica gel plate; eluent—50% EtOAc/hexanes) to give 6.4 mg of an oily residue.

¹H NMR (400 MHz, CD₃OD) δ ppm 1.04 (s, 5H), 1.08-1.16 (m, 6H), 1.40-1.45 (s, 4H), 1.94-2.09 (m, 2H), 2.50-2.60 (m, 1H), 3.5-3.72 (m, 1H), 3.84-4.04 (m, 1H), 4.28-4.42 (m, 1H), 4.50-4.70 (m, 1H), 5.20-5.36 (m, 1H), 7.34-7.67 (m, 8H), 8.23-8.34 (m, 1H); +APCI MS (M+1) 577.2.

Step 2: A solution of the product obtained in Step 1 in absolute ethanol (150 μL) and concentrated HCl (75 μL) was stirred for 17 hours at room temperature and concentrated in vacuo. The residue was diluted with ethanol and concentrated to give 4.5 mg of (7A) as a residual, colorless glass.

¹H NMR (400 MHz, CD₃OD) δ ppm 1.09-1.16 (two overlapping doublets, 6H), 2.20-2.34 (m, 2H), 2.52-2.62 (m, 1H), 3.50-3.74 (m, 2H), 4.23-4.34 (m, 2H), 5.32-5.40 (m, 2H), 7.38-7.50 (m, 6H), 7.65-7.70 (m, 2H); +APCI MS (M+1) 477.2 (M+1).

The compounds listed in Table 5 were prepared utilizing procedures analogous to those described above for the preparation of Compound 7A using the appropriate starting materials.

TABLE 5

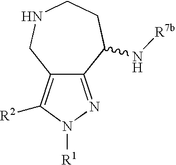

| Ex. | | | | | Mass Spec |
|---|---|---|---|---|---|
| No. | R² | R¹ | R⁴ | R^{7b} | m/z (M + 1) |
| 7B | 4-chloro- | 2-chloro- | H | —C(O)—CH(CH₃)₂ | 443.2 |
| | phenyl | phenyl | | | |
| 7C | 4-chloro- | 2-chloro- | H | —C(O)—N(CH₂CH₃)₂ | 472.2 |
| | phenyl | phenyl | | | |
| 7D | 4-chloro- | 2-chloro- | H | —C(O)—OCH(CH₃)₂ | 459.2 |
| | phenyl | phenyl | | | |

### Pharmacological Testing

The utility of the compounds of the present invention in the practice of the instant invention can be evidenced by activity in at least one of the protocols described hereinbelow. The following acronyms are used in the protocols described below.

BSA—bovine serum albumin

DMSO—dimethylsulfoxide

EDTA—ethylenediamine tetracetic acid

PBS—phosphate-buffered saline

EGTA—ethylene glycol-bis(β-aminoethyl ether) N,N,N′,N′-tetraacetic acid

GDP—guanosine diphosphate

sc—subcutaneous

po—orally

ip—intraperitoneal

icv—intra cerebro ventricular

iv—intravenous

[³H]SR141716A—radiolabeled N-(piperidin-1-yl)-5-(4-chlorophenyl)-1-(2,4-dichlorophenyl)-4-methyl-1H-pyrazole-3-carboxamide hydrochloride available from Amersham Biosciences, Piscataway, N.J.

[³H]CP-55940—radiolabled 5-(1,1-dimethylheptyl)-2-[5-hydroxy-2-(3-hydroxypropyl)-cyclohexyl]-phenol available from NEN Life Science Products, Boston, Mass.

AM251—N-(piperidin-1-yl)-1-(2,4-dichlorophenyl)-5-(4-iodophenyl)-4-methyl-1H-pyrazole-3-carboxamide available from Tocris™, Ellisville, Mo.

Compounds having an activity <20 nM are generally tested in the CB-1 GTPγ [³⁵S] Binding Assay and the CB-2 binding assay described below in the Biological Binding Assays section. Selected compounds are then tested in vivo using one or more of the functional assays described in the Biological Functional Assays section below. The compounds in the Examples above provided a range of CB-1 receptor binding activities from 0.1 to 500 nM.

Example 1C provided a binding activity of 47 nM.

### In Vitro Biological Assays

Bioassay systems for determining the CB-1 and CB-2 binding properties and pharmacological activity of cannabinoid receptor ligands are described by Roger G. Pertwee in “Pharmacology of Cannabinoid Receptor Ligands” Current Medicinal Chemistry, 6, 635-664 (1999) and in WO 92/02640 (U.S. application Ser. No. 07/564,075 filed Aug. 8, 1990, incorporated herein by reference).

The following assays were designed to detect compounds that inhibit the binding of [³H] SR141716A (selective radiolabeled CB-1 ligand) and [³H]5-(1,1-dimethylheptyl)-2-[5-hydroxy-2-(3-hydroxypropyl)-cyclohexyl]-phenol; radiolabeled CB-1/CB-2 ligand) to their respective receptors.

### Rat CB-1 Receptor Binding Protocol

PelFreeze brains (available from Pel Freeze Biologicals, Rogers, Ark.) were cut up and placed in tissue preparation buffer (5 mM Tris HCl, pH=7.4 and 2 mM EDTA), polytroned at high speed and kept on ice for 15 minutes. The homogenate was then spun at 1,000×G for 5 minutes at 4° C. The supernatant was recovered and centrifuged at 100,000×G for 1 hour at 4° C. The pellet was then re-suspended in 25 mL of TME (25 nM Tris, pH=7.4, 5 mM MgCl₂, and 1 mM EDTA) per brain used. A protein assay was performed and 200 μl of tissue totaling 20 μg was added to the assay.

The test compounds were diluted in drug buffer (0.5% BSA, 10% DMSO and TME) and then 25 μl were added to a deep well polypropylene plate. [³H] SR141716A was diluted in a ligand buffer (0.5% BSA plus TME) and 25 μl were added to the plate. A BCA protein assay was used to determine the appropriate tissue concentration and then 200 μl of rat brain tissue at the appropriate concentration was added to the plate. The plates were covered and placed in an incubator at 20° C. for 60 minutes. At the end of the incubation period 250 JA of stop buffer (5% BSA plus TME) was added to the reaction plate. The plates were then harvested by Skatron onto GF/B filtermats presoaked in BSA (5 mg/mL) plus TME. Each filter was washed twice. The filters were dried overnight. In the morning the filters were counted on a Wallac Betaplate™ counter (available from PerkinElmer Life Sciences™, Boston, Mass.).

### Human CB-1 Receptor Binding Protocol

Human embryonic kidney 293 (HEK 293) cells transfected with the CB-1 receptor cDNA (obtained from Dr. Debra Kendall, University of Connecticut) were harvested in homogenization buffer (10 mM EDTA, 10 mM EGTA, 10 mM Na Bicarbonate, protease inhibitors; pH=7.4), and homogenized with a Dounce Homogenizer. The homogenate was then spun at 1,000×g for 5 minutes at 4° C. The supernatant was recovered and centrifuged at 25,000×G for 20 minutes at 4° C. The pellet was then re-suspended in 10 mL of homogenization buffer and re-spun at 25,000×G for 20 minutes at 4° C. The final pellet was re-suspended in 1 mL of TME (25 mM Tris buffer (pH=7.4) containing 5 mM MgCl₂ and 1 mM EDTA). A protein assay was performed and 200 μl of tissue totaling 20 μg was added to the assay.

The test compounds were diluted in drug buffer (0.5% BSA, 10% DMSO and TME) and then 25 μl were added to a deep well polypropylene plate. [³H] SR141716A was diluted in a ligand buffer (0.5% BSA plus TME) and 25 μl were added to the plate. The plates were covered and placed in an incubator at 30° C. for 60 minutes. At the end of the incubation period 250 μl of stop buffer (5% BSA plus TME) was added to the reaction plate. The plates were then harvested by Skatron onto GF/B filtermats presoaked in BSA (5 mg/mL) plus TME. Each filter was washed twice. The filters were dried overnight. In the morning the filters were counted on a Wallac Betaplate™ counter (available from PerkinElmer Life Sciences™, Boston, Mass.).

### CB-2 Receptor Binding Protocol

Chinese hamster ovary-K1 (CHO-K1) cells transfected with CB-2 cDNA (obtained from Dr. Debra Kendall, University of Connecticut) were harvested in tissue preparation buffer (5 mM Tris-HCl buffer (pH=7.4) containing 2 mM EDTA), polytroned at high speed and kept on ice for 15 minutes. The homogenate was then spun at 1,000×G for 5 minutes at 4° C. The supernatant was recovered and centrifuged at 100,000×G for 1 hour at 4° C. The pellet was then re-suspended in 25 mL of TME (25 mM Tris buffer (pH=7.4) containing 5 mM MgCl₂ and 1 mM EDTA) per brain used. A protein assay was performed and 200 μl of tissue totaling 10 μg was added to the assay.

The test compounds were diluted in drug buffer (0.5% BSA, 10% DMSO, and 80.5% TME) and then 25 μl were added to the deep well polypropylene plate. [³H]5-(1,1-Dimethyl-heptyl)-2-[5-hydroxy-2-(3-hydroxy-propyl)-cyclohexyl]-phenol was diluted a ligand buffer (0.5% BSA and 99.5% TME) and then 25 μl were added to each well at a concentration of 1 nM. A BCA protein assay was used to determine the appropriate tissue concentration and 200 μl of the tissue at the appropriate concentration was added to the plate. The plates were covered and placed in an incubator at 30° C. for 60 minutes. At the end of the incubation period 250 μl of stop buffer (5% BSA plus TME) was added to the reaction plate. The plates were then harvested by Skatron format onto GF/B filtermats presoaked in BSA (5 mg/mL) plus TME. Each filter was washed twice. The filters were dried overnight. The filters were then counted on the Wallac Betaplate™ counter.

### CB-1 GTPγ [³⁵S] Binding Assay

Membranes were prepared from CHO-K1 cells stably transfected with the human CB-1 receptor cDNA. Membranes were prepared from cells as described by Bass et al, in “Identification and characterization of novel somatostatin antagonists,” Molecular Pharmacology, 50, 709-715 (1996). GTPγ [³⁵S] binding assays were performed in a 96 well FlashPlate™ format in duplicate using 100 pM GTPγ[³⁵S] and 10 μg membrane per well in assay buffer composed of 50 mM Tris HCl, pH 7.4, 3 mM MgCl₂, pH 7.4, 10 mM MgCl₂, 20 mM EGTA, 100 mM NaCl, 30 μM GDP, 0.1% bovine serum albumin and the following protease inhibitors: 100 μg/mL bacitracin, 100 μg/mL benzamidine, 5 μg/mL aprotinin, 5 μg/mL leupeptin. The assay mix was then incubated with increasing concentrations of antagonist (10⁻¹⁰ M to 10⁻⁵ M) for 10 minutes and challenged with the cannabinoid agonist 5-(1,1-dimethyl-heptyl)-2-[5-hydroxy-2-(3-hydroxy-propyl)-cyclohexyl]-phenol (10 μM). Assays were performed at 30° C. for one hour. The FlashPlates™ were then centrifuged at 2000×G for 10 minutes. Stimulation of GTPγ[³⁵S] binding was then quantified using a Wallac Microbeta.EC₅₀ calculations done using Prism™ by Graphpad.

Inverse agonism was measured in the absense of agonist.

### CB-1 FLIPR-Based Functional Assay Protocol

CHO-K1 cells co-transfected with the human CB-1 receptor cDNA (obtained from Dr. Debra Kendall, University of Connecticut) and the promiscuous G-protein G16 were used for this assay. Cells were plated 48 hours in advance at 12500 cells per well on collagen coated 384 well black clear assay plates. Cells were incubated for one hour with 4 μM Fluo-4 AM (Molecular Probes) in DMEM (Gibco) containing 2.5 mM probenicid and pluronic acid (0.04%). The plates were then washed 3 times with HEPES-buffered saline (containing probenicid; 2.5 mM) to remove excess dye. After 20 min the plates were added to the FLIPR individually and fluorescence levels was continuously monitored over an 80 second period. Compound additions were made simultaneously to all 384 wells after 20 seconds of baseline. Assays were performed in triplicate and 6 point concentration-response curves generated. Antagonist compounds were subsequently challenged with 3 μM WIN 55,212-2 (agonist). Data were analyzed using Graph Pad Prism.

### Detection of Inverse Agonists

The following cyclic-AMP assay protocol using intact cells was used to determine inverse agonist activity.

Cells were plated into a 96-well plate at a plating density of 10,000-14,000 cells per well at a concentration of 100 μl per well. The plates were incubated for 24 hours in a 37° C. incubator. The media was removed and media lacking serum (100 μl) was added. The plates were then incubated for 18 hours at 37° C.

Serum free medium containing 1 mM IBMX was added to each well followed by 10 μl of test compound (1:10 stock solution (25 mM compound in DMSO) into 50% DMSO/PBS) diluted 10× in PBS with 0.1% BSA. After incubating for 20 minutes at 37° C., 2 μM of Forskolin was added and then incubated for an additional 20 minutes at 37° C. The media was removed, 100 μl of 0.01 N HCl was added and then incubated for 20 minutes at room temperature. Cell lysate (75 μl) along with 25 μl of assay buffer (supplied in FlashPlate™ cAMP assay kit available from NEN Life Science Products Boston, Mass.) into a Flashplate. cAMP standards and cAMP tracer were added following the kit's protocol. The flashplate was then incubated for 18 hours at 4° C. The content of the wells were aspirated and counted in a Scintillation counter.

### In Vivo Biological Assays

Cannabinoid agoinists such as Δ⁹-tetrahydrocannabinol (Δ⁹-THC) and 5-(1,1-dimethyl-heptyl)-2-[5-hydroxy-2-(3-hydroxy-propyl)-cyclohexyl]-phenolhave been shown to affect four characteristic behaviors in mice, collectively known as the Tetrad. For a description of these behaviors see: Smith, P. B., et al. in “The pharmacological activity of anandamide, a putative endogenous cannabinoid, in mice.” J. Pharmacol. Exp. Ther., 270(1), 219-227 (1994) and Wiley, J., et al. in “Discriminative stimulus effects of anandamide in rats,” Eur. J. Pharmacol., 276(1-2), 49-54 (1995). Reversal of these activities in the Locomotor Activity, Catalepsy, Hypothermia, and Hot Plate assays described below provides a screen for in vivo activity of CB-1 antagonists.

All data is presented as % reversal from agonist alone using the following formula: (5-(1,1-dimethyl-heptyl)-2-[5-hydroxy-2-(3-hydroxy-propyl)-cyclohexyl]-phenol/agonist−vehicle/agonist)/(vehicle/vehicle−vehicle/agonist). Negative numbers indicate a potentiation of the agonist activity or non-antagonist activity. Positive numbers indicate a reversal of activity for that particular test.

### Locomotor Activity

Male ICR mice (n=6; 17-19 g, Charles River Laboratories, Inc., Wilmington, Mass.) were pre-treated with test compound (sc, po, ip, or icv). Fifteen minutes later, the mice were challenged with 5-(1,1-dimethyl-heptyl)-2-[5-hydroxy-2-(3-hydroxy-propyl)-cyclohexyl]-phenol (sc). Twenty-five minutes after the agonist injection, the mice were placed in clear acrylic cages (431.8 cm×20.9 cm×20.3 cm) containing clean wood shavings. The subjects were allowed to explore surroundings for a total of about 5 minutes and the activity was recorded by infrared motion detectors (available from Coulbourn Instruments™, Allentown, Pa.) that were placed on top of the cages. The data was computer collected and expressed as “movement units.”

### Catalepsy

Male ICR mice (n=6; 17-19 g upon arrival) were pre-treated with test compound (sc, po, ip or icv). Fifteen minutes later, the mice were challenged with 5-(1,1-dimethyl-heptyl)-2-[5-hydroxy-2-(3-hydroxy-propyl)-cyclohexyl]-phenol (sc). Ninety minutes post injection, the mice were placed on a 6.5 cm steel ring attached to a ring stand at a height of about 12 inches. The ring was mounted in a horizontal orientation and the mouse was suspended in the gap of the ring with fore- and hind-paws gripping the perimeter. The duration that the mouse remained completely motionless (except for respiratory movements) was recorded over a 3-minute period.

The data were presented as a percent immobility rating. The rating was calculated by dividing the number of seconds the mouse remains motionless by the total time of the observation period and multiplying the result by 100. A percent reversal from the agonist was then calculated.

### Hypothermia

Male ICR mice (n=5; 17-19 g upon arrival) were pretreated with test compounds (sc, po, ip or icv). Fifteen minutes later, mice were challenged with the cannabinoid agonist 5-(1,1-dimethyl-heptyl)-2-[5-hydroxy-2-(3-hydroxy-propyl)-cyclohexyl]-phenol (sc). Sixty-five minutes post agonist injection, rectal body temperatures were taken. This was done by inserting a small thermostat probe approximately 2-2.5 cm into the rectum. Temperatures were recorded to the nearest tenth of a degree

### Hot Plate

Male ICR mice (n=7; 17-19 g upon arrival) are pre-treated with test compounds (sc, po, ip or iv). Fifteen minutes later, mice were challenged with a cannabinoid agonist 5-(1,1-dimethyl-heptyl)-2-[5-hydroxy-2-(3-hydroxy-propyl)-cyclohexyl]-phenol (sc). Forty-five minutes later, each mouse was tested for reversal of analgesia using a standard hot plate meter (Columbus Instruments). The hot plate was 10″×10″×0.75″ with a surrounding clear acrylic wall. Latency to kick, lick or flick hindpaw or jump from the platform was recorded to the nearest tenth of a second. The timer was experimenter activated and each test had a 40 second cut off. Data were presented as a percent reversal of the agonist induced analgesia.

### Food Intake

The following screen was used to evaluate the efficacy of test compounds for inhibiting food intake in Sprague-Dawley rats after an overnight fast.

Male Sprague-Dawley rats were obtained from Charles River Laboratories, Inc. (Wilmington, Mass.). The rats were individually housed and fed powdered chow. They were maintained on a 12-hour light/dark cycle and received food and water ad libitum. The animals were acclimated to the vivarium for a period of one week before testing was conducted. Testing was completed during the light portion of the cycle.

To conduct the food intake efficacy screen, rats were transferred to individual test cages without food the afternoon prior to testing, and the rats were fasted overnight. After the overnight fast, rats were dosed the following morning with vehicle or test compounds. A known antagonist was dosed (3 mg/kg) as a positive control, and a control group received vehicle alone (no compound). The test compounds were dosed at ranges between 0.1 and 100 mg/kg depending upon the compound. The standard vehicle was 0.5% (w/v) methylcellulose in water and the standard route of administration was oral. However, different vehicles and routes of administration were used to accommodate various compounds when required. Food was provided to the rats 30 minutes after dosing and the Oxymax automated food intake system (Columbus Instruments, Columbus, Ohio) was started. Individual rat food intake was recorded continuously at 10-minute intervals for a period of two hours. When required, food intake was recorded manually using an electronic scale; food was weighed every 30 minutes after food was provided up to four hours after food was provided. Compound efficacy was determined by comparing the food intake pattern of compound-treated rats to vehicle and the standard positive control.

### Alcohol Intake

The following protocol evaluates the effects of alcohol intake in alcohol preferring (P) female rats (bred at Indiana University) with an extensive drinking history. The following references provide detailed descriptions of P rats: Li, T.-K., et al., “Indiana selection studies on alcohol related behaviors” in Development of Animal Models as Pharmacogenetic Tools (eds McClearn C. E., Deitrich R. A. and Erwin V. G.), Research Monograph 6, 171-192 (1981) NIAAA, ADAMHA, Rockville, Md.; Lumeng, L, et al., “New strains of rats with alcohol preference and nonpreference” Alcohol And Aldehyde Metabolizing Systems, 3, Academic Press, New York, 537-544 (1977); and Lumeng, L, et al., “Different sensitivities to ethanol in alcohol-preferring and -nonpreferring rats,” Pharmacol. Biochem Behav., 16, 125-130 (1982).

Female rats were given 2 hours of access to alcohol (10% v/v and water, 2-bottle choice) daily at the onset of the dark cycle. The rats were maintained on a reverse cycle to facilitate experimenter interactions. The animals were initially assigned to four groups equated for alcohol intakes: Group 1—vehicle (n=8); Group 2—positive control (e.g., 5.6 mg/kg AM251; n=8); Group 3—low dose test compound (n=8); and Group 4—high dose of test compound (n=8). Test compounds were generally mixed into a vehicle of 30% (w/v) β-cyclodextrin in distilled water at a volume of 1-2 mL/kg. Vehicle injections were given to all groups for the first two days of the experiment. This was followed by 2 days of drug injections (to the appropriate groups) and a final day of vehicle injections. On the drug injection days, drugs were given sc 30 minutes prior to a 2-hour alcohol access period. Alcohol intake for all animals was measured during the test period and a comparison was made between drug and vehicle-treated animals to determine effects of the compounds on alcohol drinking behavior.

Additional drinking studies were done utilizing female C57BL/6 mice (Charles River). Several studies have shown that this strain of mice will readily consume alcohol with little to no manipulation required (Middaugh et al., “Ethanol Consumption by C57BL/6 Mice: Influence of Gender and Procedural Variables” Alcohol, 17 (3), 175-183, 1999; Le et al., “Alcohol Consumption by C57BL/6, BALA/c, and DBA/2 Mice in a Limited Access Paradigm” Pharmacology Biochemisrty and Behavior, 47, 375-378, 1994).

For our purposes, upon arrival (17-19 g) mice were individually housed and given unlimited access to powdered rat chow, water and a 10% (w/v) alcohol solution. After 2-3 weeks of unlimited access, water was restricted for 20 hours and alcohol was restricted to only 2 hours access daily. This was done in a manner that the access period was the last 2 hours of the dark part of the light cycle.

Once drinking behavior stabilized, testing commenced. Mice were considered stable when the average alcohol consumption for 3 days was ±20% of the average for all 3 days. Day 1 of test consisted of all mice receiving vehicle injection (sc or ip). Thirty to 120 minutes post injection access was given to alcohol and water. Alcohol consumption for that day was calculated (g/kg) and groups were assigned (n=7-10) so that all groups had equivocal alcohol intake. On day 2 and 3, mice were injected with vehicle or drug and the same protocol as the previous day was followed. Day 4 was wash out and no injections were given. Data was analyzed using repeated measures ANOVA. Change in water or alcohol consumption was compared back to vehicle for each day of the test. Positive results would be interpreted as a compound that was able to significantly reduce alcohol consumption while having no effect on water

### Oxygen Consumption

Methods:

Whole body oxygen consumption is measured using an indirect calorimeter (Oxymax from Columbus Instruments, Columbus, Ohio) in male Sprague Dawley rats (if another rat strain or female rats are used, it will be specified). Rats (300-380 g body weight) are placed in the calorimeter chambers and the chambers are placed in activity monitors. These studies are done during the light cycle. Prior to the measurement of oxygen consumption, the rats are fed standard chow ad libitum. During the measurement of oxygen consumption, food is not available. Basal pre-dose oxygen consumption and ambulatory activity are measured every 10 minutes for 2.5 to 3 hours. At the end of the basal pre-dosing period, the chambers are opened and the animals are administered a single dose of compound (the usual dose range is 0.001 to 10 mg/kg) by oral gavage (or other route of administration as specified, i.e., sc, ip, iv). Drugs are prepared in methylcellulose, water or other specified vehicle (examples include PEG400, 30% beta-cyclo dextran and propylene glycol). Oxygen consumption and ambulatory activity are measured every 10 minutes for an additional 1-6 hours post-dosing.

The Oxymax calorimeter software calculates the oxygen consumption (mL/kg/h) based on the flow rate of air through the chambers and difference in oxygen content at inlet and output ports. The activity monitors have 15 infrared light beams spaced one inch apart on each axis, ambulatory activity is recorded when two consecutive beams are broken and the results are recorded as counts.

Resting oxygen consumption, during pre- and post-dosing, is calculated by averaging the 10-min O₂ consumption values, excluding periods of high ambulatory activity (ambulatory activity count>100) and excluding the first 5 values of the pre-dose period and the first value from the post-dose period. Change in oxygen consumption is reported as percent and is calculated by dividing the post-dosing resting oxygen consumption by the pre-dose oxygen consumption *100. Experiments will typically be done with n=4-6 rats and results reported are mean+/−SEM.

Interpretation:

An increase in oxygen consumption of >10% is considered a positive result. Historically, vehicle-treated rats have no change in oxygen consumption from pre-dose basal.

What is claimed is:

1. A compound of Formula (I) 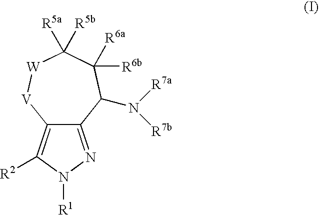
wherein
R¹ and R² are each independently an aryl optionally substituted with one or more substituents, or a heteroaryl optionally substituted with one or more substituents;
V is O and W is CR^{3a}R^{3b}, or V is CR^{3a}R^{3b} and W is N—R⁴;
R^{3a}, R^{3b}, R^{5a}, R^{5b}, R^{6a}, R^{6b}, and R^{7a} are each independently hydrogen, (C₁-C₄)alkyl, or halo-substituted (C₁-C₄)alkyl;
R⁴ is hydrogen, (C₁-C₄)alkyl, halo-substituted (C₁-C₄)alkyl, ((C₁-C₄)alkoxy)-C(O)—, aryl, ((C₁-C₄)alkyl)C(O)—, (aryl)-C(O)—, ((C₁-C₄)alkyl)-SO₂—, or (aryl)-SO₂—;
R^{7b} is
(i) hydrogen,
(ii) (C₁-C₆)alkyl,
(iii) (C₂-C₆)alkenyl,
(iv) halo-substituted (C₁-C₄)alkyl,
(v) —C(O)—(CH₂)ₚR⁸, where p is 0 or 1, and R⁸ is a chemical moiety selected from the group consisting of C₁-C₆)alkyl, (C₂-C₆)alkenyl, (C₁-C₄)alkoxy, (C₃-C₇)cycloalkyl, (C₁-C₄)alkyl)-SO₂—, 3- to 6-membered heterocycle containing one to three heteroatoms independently selected from O, N and S, 5- to 6-membered lactam or lactone, and 5- to 6-membered heteroaryl containing one to three heteroatoms independently selected from O, N and S, where said chemical moiety is optionally substituted with one or more substituents selected from (C₁-C₄)alkyl, (C₁-C₄)alkoxy, trifluoromethyl, halo, cyano, amino, (C₁-C₄)alkyl amino, or di(C₁-C₄)alkyl amino;
or R⁸ taken together with R^{7a} form a 5- to 6-membered lactam;
(vi) —C(O)—O—R⁹, where R⁹ is (C₁-C₆)alkyl, halo-substituted (C₁-C₆)alkyl or (C₁-C₄)alkoxy(C₁-C₆)alkyl,
or R⁹ taken together with R^{7a} form a 5- to 6-membered lactone;
(vii) —C(O)—N(R^{10a})(R^{10b}), where R^{10a} is hydrogen, (C₁-C₆)alkyl, or halo-substituted (C₁-C₄)alkyl, and R^{10b} is hydrogen, (C₁-C₆)alkyl, halo-substituted (C₁-C₄)alkyl, (C₂-C₆)alkenyl, (C₃-C₇)cycloalkyl, 3- to 6-membered heterocycle containing one to three heteroatoms independently selected from O, N and S, 5- to 6-membered lactam or lactone, and 5- to 6-membered heteroaryl containing one to three heteroatoms independently selected from O, N and S, where said chemical moiety is optionally substituted with one or more substituents selected from (C₁-C₄)alkyl, (C₁-C₄)alkoxy, trifluoromethyl, halo, cyano, amino, (C₁-C₄)alkyl amino, or di(C₁-C₄)alkyl amino,
or R^{10a} and R^{10b} taken together form a piperidine or pyrrolidine,
or either R^{10a} or R^{10b} taken together with R^{7a} form a 5- or 6-membered lactam;
a pharmaceutically acceptable salt thereof, or a solvate or hydrate of the compound or the salt.

2. The compound of claim 1 wherein V is oxygen, W is CR^{3a}R^{3b}, and R^{7a} and R^{7b} are each independently hydrogen, (C₁-C₆)alkyl, (C₂-C₆)alkenyl, or halo-substituted (C₁-C₄)alkyl; or a pharmaceutically acceptable salt thereof, or a solvate or hydrate of the compound or the salt.

3. A compound of claim 2 selected from the group consisting of
3-(4-Chloro-phenyl)-2-(2-chloro-phenyl)-5,6,7,8-tetrahydro-2H-4-oxa-1,2-diaza-azulen-8-ylamine;
[3-(4-Chloro-phenyl)-2-(2-chloro-phenyl)-5,6,7,8-tetrahydro-2H-4-oxa-1,2-diaza-azulen-8-yl]-(2,2,2-trifluoro-ethyl)-amine;
[3-(4-Chloro-phenyl)-2-(2-chloro-phenyl)-5,6,7,8-tetrahydro-2H-4-oxa-1,2-diaza-azulen-8-yl]-methyl-amine; and
Allyl-[3-(4-chloro-phenyl)-2-(2-chloro-phenyl)-5,6,7,8-tetrahydro-2H-4-oxa-1,2-diaza-azulen-8-yl]-amine;
or a pharmaceutically acceptable salt thereof, or a solvate or hydrate of the compound or the salt.

4. The compound of claim 1 wherein V is oxygen, W is CR^{3a}R^{3b}, and R^{7b} is —C(O)—(CH₂)ₚR⁸; or a pharmaceutically acceptable salt thereof, or a solvate or hydrate of said compound or said salt.

5. A compound of claim 4 selected from the group consisting of
N-[3-(4-Chloro-phenyl)-2-(2-chloro-phenyl)-5,6,7,8-tetrahydro-2H-4-oxa-1,2-diaza-azulen-8-yl]-acetamide;
Cyclopentanecarboxylic acid [3-(4-chloro-phenyl)-2-(2-chloro-phenyl)-5,6,7,8-tetrahydro-2H-4-oxa-1,2-diaza-azulen-8-yl]-amide;
N-[3-(4-Chloro-phenyl)-2-(2-chloro-phenyl)-5,6,7,8-tetrahydro-2H-4-oxa-1,2-diaza-azulen-8-yl]-isobutyramide;
N-[3-(4-Chloro-phenyl)-2-(2-chloro-phenyl)-5,6,7,8-tetrahydro-2H-4-oxa-1,2-diaza-azulen-8-yl]-propionamide;
Cyclobutanecarboxylic acid [3-(4-chloro-phenyl)-2-(2-chloro-phenyl)-5,6,7,8-tetrahydro-2H-4-oxa-1,2-diaza-azulen-8-yl]-amide;
N-[3-(4-Chloro-phenyl)-2-(2-chloro-phenyl)-5,6,7,8-tetrahydro-2H-4-oxa-1,2-diaza-azulen-8-yl]-2,2-dimethyl-propionamide;
Cyclopropanecarboxylic acid [3-(4-chloro-phenyl)-2-(2-chloro-phenyl)-5,6,7,8-tetrahydro-2H-4-oxa-1,2-diaza-azulen-8-yl]-amide;
N-[3-(4-Chloro-phenyl)-2-(2-chloro-phenyl)-5,6,7,8-tetrahydro-2H-4-oxa-1,2-diaza-azulen-8-yl]-isobutyramide;
N-[2-(2-Chloro-phenyl)-3-(4-cyano-phenyl)-5,6,7,8-tetrahydro-2H-4-oxa-1,2-diaza-azulen-8-yl]4,4,4-trifluoro-butyramide;
N-[3-(4-Chloro-phenyl)-2-(2-chloro-phenyl)-5,6,7,8-tetrahydro-2H-4-oxa-1,2-diaza-azulen-8-yl]-3,3-dimethyl-butyramide; and
1-[3-(4-Chloro-phenyl)-2-(2-chloro-phenyl)-5,6,7,8-tetrahydro-2H-4-oxa-1,2-diaza-azulen-8-yl]-pyrrolidin-2-one;
or a pharmaceutically acceptable salt thereof, or a solvate or hydrate of said compound or said salt.

6. The compound of claim 1 wherein V ix oxygen, W is CR^{3a}R^{3b}, and R^{7b} is —C(O)—O—R⁹;
or a pharmaceutically acceptable salt thereof, or a solvate or hydrate of the compound or the salt.

7. A compound of claim 6 selected from the group consisting of
[3-(4-Chloro-phenyl)-2-(2-chloro-phenyl)-5,6,7,8-tetrahydro-2H-4-oxa-1,2-diaza-azulen-8-yl]-carbamic acid isopropyl ester;
[3-(4-Chloro-phenyl)-2-(2-chloro-phenyl)-5,6,7,8-tetrahydro-2H-4-oxa-1,2-diaza-azulen-8-yl]-carbamic acid methyl ester;
[3-(4-Chloro-phenyl)-2-(2-cyano-phenyl)-5,6,7,8-tetrahydro-2H-4-oxa-1,2-diaza-azulen-8-yl]-carbamic acid tert-butyl ester;
[2-(2-Chloro-phenyl)-3-(4-cyano-phenyl)-5,6,7,8-tetrahydro-2H-4-oxa-1,2-diaza-azulen-8-yl]-carbamic acid ethyl ester;
[2-(2-Chloro-phenyl)-3-(4-cyano-phenyl)-5,6,7,8-tetrahydro-2H-4-oxa-1,2-diaza-azulen-8-yl]-carbamic acid isopropyl ester;
[2-(2-Chloro-phenyl)-3-(4-cyano-phenyl)-5,6,7,8-tetrahydro-2H-4-oxa-1,2-diaza-azulen-8-yl]-carbamic acid propyl ester;
[3-(4-Chloro-phenyl)-2-(2-chloro-phenyl)-5,6,7,8-tetrahydro-2H-4-oxa-1,2-diaza-azulen-8-yl]-carbamic acid 2-methoxy-ethyl ester;
[3-(4-Chloro-phenyl)-2-(2-chloro-phenyl)-5,6,7,8-tetrahydro-2H-4-oxa-1,2-diaza-azulen-8-yl]-carbamic acid propyl ester;
[3-(4-Chloro-phenyl)-2-(2-chloro-phenyl)-5,6,7,8-tetrahydro-2H-4-oxa-1,2-diaza-azulen-8-yl]-carbamic acid ethyl ester; and
[3-(4-Chloro-phenyl)-2-(2-chloro-phenyl)-5,6,7,8-tetrahydro-2H-4-oxa-1,2-diaza-azulen-8-yl]-carbamic acid tert-butyl ester;
or a pharmaceutically acceptable salt thereof, or a solvate or hydrate of said compound or said salt.

8. The compound of claim 1 wherein V is oxygen, W is CR^{3a}R^{3b}, and R^{7b} is —C(O)—N(R^{10a})(R^{10b});
or a pharmaceutically acceptable salt thereof, or a solvate or hydrate of the compound or the salt.

9. A compound of claim 8 selected from the group consisting of
1-[3-(4-Chloro-phenyl)-2-(2-chloro-phenyl)-5,6,7,8-tetrahydro-2H-4-oxa-1,2-diaza-azulen-8-yl]-3-ethyl-urea;
3-[3-(4-Chloro-phenyl)-2-(2-chloro-phenyl)-5,6,7,8-tetrahydro-2H-4-oxa-1,2-diaza-azulen-8-yl]-1,1-diethyl-urea;
1-[2-(2-Chloro-phenyl)-3-(4-cyano-phenyl)-5,6,7,8-tetrahydro-2H-4-oxa-1,2-diaza-azulen-8-yl]-3-ethyl-urea;
1-[2-(2-Chloro-phenyl)-3-(4-cyano-phenyl)-5,6,7,8-tetrahydro-2H-4-oxa-1,2-diaza-azulen-8-yl]-3-propyl-urea;
1-[2-(2-Chloro-phenyl)-3-(4-cyano-phenyl)-5,6,7,8-tetrahydro-2H-4-oxa-1,2-diaza-azulen-8-yl]-3-isopropyl-urea;
1-[2-(2-Chloro-phenyl)-3-(4-cyano-phenyl)-5,6,7,8-tetrahydro-2H-4-oxa-1,2-diaza-azulen-8-yl]-3-cyclopentyl-urea;
1-tert-Butyl-3-[2-(2-chloro-phenyl)-3-(4-cyano-phenyl)-5,6,7,8-tetrahydro-2H-4-oxa-1,2-diaza-azulen-8-yl]-urea;
1-[3-(4-Chloro-phenyl)-2-(2-chloro-phenyl)-5,6,7,8-tetrahydro-2H-4-oxa-1,2-diaza-azulen-8-yl]-3-cyclopentyl-urea;
1-tert-Butyl-3-[3-(4-chloro-phenyl)-2-(2-chloro-phenyl)-5,6,7,8-tetrahydro-2H-4-oxa-1,2-diaza-azulen-8-yl]-urea;
1-[3-(4-Chloro-phenyl)-2-(2-chloro-phenyl)-5,6,7,8-tetrahydro-2H-4-oxa-1,2-diaza-azulen-8-yl]-3-isopropyl-urea;
1-[3-(4-Chloro-phenyl)-2-(2-chloro-phenyl)-5,6,7,8-tetrahydro-2H-4-oxa-1,2-diaza-azulen-8-yl]-3-propyl-urea;
1-sec-Butyl-3-[3-(4-chloro-phenyl)-2-(2-chloro-phenyl)-5,6,7,8-tetrahydro-2H-4-oxa-1,2-diaza-azulen-8-yl]-urea; and
Pyrrolidine-1-carboxylic acid [3-(4-chloro-phenyl)-2-(2-chloro-phenyl)-5,6,7,8-tetrahydro-2H-4-oxa-1,2-diaza-azulen-8-yl]-amide;
or a pharmaceutically acceptable salt thereof, or a solvate or hydrate of the compound or the salt.

10. The compound of claim 1 wherein R¹ and R² are each independently a chemical moiety selected from phenyl, thiophenyl, pyridyl or pyrimidinyl, where said chemical moiety is substituted with one or more substituents;
or a pharmaceutically acceptable salt thereof, or a solvate or hydrate of said compound or said salt.

11. The compound of claim 10 wherein R¹ is a phenyl substituted with one to three substituents independently selected from the group consisting of halo, (C₁-C₄)alkoxy, (C₁-C₄)alkyl, fluoro-substituted (C₁-C₄)alkyl, and cyano; and
R² is phenyl, pyridyl, thiophenyl, or pyrimidinyl, where said phenyl, said pyridyl, said thienyl, and said pyrimidinyl are each substituted with one to three substituents independently selected from the group consisting of halo, (C₁-C₄)alkoxy, (C₁-C₄)alkyl, fluoro-substituted (C₁-C₄)alkyl, and cyano;
or a pharmaceutically acceptable salt thereof, or a solvate or hydrate of said compound or said salt.

12. The compound of claim 11 wherein R¹ is 2-chlorophenyl, 2-fluorophenyl, 2-bromophenyl, 2-cyanophenyl, 2,4-dichlorophenyl, 4-chloro-2-fluorophenyl, 2-chloro-4-fluorophenyl, 2-methylphenyl, 2-chloro-4-methylphenyl, or 2,4-difluorophenyl; and
R² is 4-chlorophenyl, 4-cyanophenyl, 4-methylphenyl, 4-ethylphenyl, 4-isopropylphenyl, 4-methoxyphenyl, 4-ethoxyphenyl, 4-isopropoxyphenyl, 4-trifluoromethylphenyl, 4-fluorophenyl, 4-bromophenyl, 6-methylpyridin-3-yl, 6-ethylpyridin-3-yl, 6-methoxypyridin-3-yl, 5-chloropyridin-2-yl, 5-trifluoromethylpyridin-2-yl, 5-methylpyridin-2-yl, 5-chlorothiophen-2-yl, or 2,4-dimethoxypyrimidin-5-yl;
or a pharmaceutically acceptable salt thereof, or a solvate or hydrate of said compound or said salt.

13. The compound of claim 1 having Formula (II) 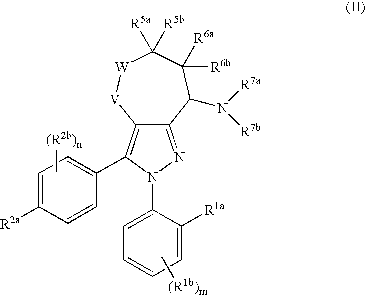
wherein
V, W, R^{5a}, R^{5b}, R^{6a}, R^{6b}, R^{7a} and R^{7b} are as defined in claim 1;
R^{1a}, R^{1b}, R^{2b}, and R^{2c} are each independently halo, (C₁-C₄)alkoxy, (C₁-C₄)alkyl, halo-substituted (C₁-C₄)alkyl, or cyano; and
n and m are each independently 0, 1 or 2;
a pharmaceutically acceptable salt thereof, or a solvate or hydrate of said compound or said salt.

14. The compound of claim 13 wherein R^{1a} is chloro, fluoro, or methyl; R^{1b} is chloro, fluoro, or methyl; R^{2a} is chloro, fluoro, (C₁-C₄)alkyl, trifluoromethyl, (C₁-C₄)alkoxy, or cyano; m is 0 or 1; and n is 0;
a pharmaceutically acceptable salt thereof, or a solvate or hydrate of said compound or said salt.

15. A pharmaceutical composition comprising (1) a compound of claim 1, or a solvate or hydrate of said compound or said salt; and (2) a pharmaceutically acceptable excipient, diluent, or carrier.

16. The composition of claim 15 further comprising at least one additional pharmaceutical agent.

17. The composition of claim 16 wherein said additional pharmaceutical agent is selected from the group consisting of a nicotine receptor partial agonist, an opioid antagonist, a dopaminergic agent, an attention deficit activity disorder agent, and an anti-obesity agents.

18. The composition of claim 17 wherein said anti-obesity agent is selected from the group consisting of an apo-B/MTP inhibitor, an 11β-hydroxy steroid dehydrogenase-1 inhibitor, peptide YY₃₋₃₆ or an analog thereof, a MCR4 agonist, a CCK-A agonist, a monoamine reuptake inhibitor, a sympathomimetic agent, a β₃ adrenergic receptor agonist, a dopamine agonist, a melanocyte-stimulating hormone receptor analog, a 5-HT2c receptor agonist, a melanin concentrating hormone antagonist, leptin, a leptin analog, a leptin receptor agonist, a galanin antagonist, a lipase inhibitor, a bombesin agonist, a neuropeptide-Y receptor antagonist, a thyromimetic agent, dehydroepiandrosterone or analog thereof, a glucocorticoid receptor antagonist, an orexin receptor antagonist, a glucagon-like peptide-1 receptor agonist, a ciliary neurotrophic factor, a human agouti-related protein antagonist, a ghrelin receptor antagonist, a histamine 3 receptor antagonist or inverse agonist, and a neuromedin U receptor agonist.

19. A method for treating a disease, condition or disorder which is modulated by a cannabinoid receptor antagonist in animals comprising the step of administering to an animal in need of such treatment a therapeutically effective amount of a compound of claim 1.

20. The method of claim 19 wherein said compound is administered in combination with a nicotine receptor partial agonist, an opioid antagonist, a dopaminergic agent, an attention deficit disorder agent, or an anti-obesity agent.

21. The method of claim 20 wherein said anti-obesity agent is selected from the group consisting of an apo-B/MTP inhibitor, an 11β-hydroxy steroid dehydrogenase-1 inhibitor, peptide YY₃₋₃₆ or an analog thereof, a MCR-4 agonist, a CCK-A agonist, a monoamine reuptake inhibitor, a sympathomimetic agent, a β₃ adrenergic receptor agonist, a dopamine agonist, a melanocyte-stimulating hormone receptor analog, a 5-HT2c receptor agonist, a melanin concentrating hormone antagonist, leptin, a leptin analog, a leptin receptor agonist, a galanin antagonist, a lipase inhibitor, a bombesin agonist, a neuropeptide-Y receptor antagonist, a thyromimetic agent, dehydroepiandrosterone or analog thereof, a glucocorticoid receptor antagonist, an orexin receptor antagonist, a glucagon-like peptide-1 receptor agonist, a ciliary neurotrophic factor, a human agouti-related protein antagonist, a ghrelin receptor antagonist, a histamine 3 receptor antagonist or inverse agonist, and a neuromedin U receptor agonist.

22. The method of claim 19 wherein said disease, condition or disorder modulated by a cannabinoid receptor antagonist is selected from the group consisting of eating disorders, weight loss or control, obesity, depression, atypical depression, bipolar disorders, psychoses, schizophrenia, behavioral addictions, suppression of reward-related behaviors, substance abuse, addictive disorders, impulsivity, alcoholism, tobacco abuse, dementia, sexual dysfunction in males, seizure disorders, epilepsy, inflammation, gastrointestinal disorders, attention deficit activity disorder, Parkinson's disease, and type II diabetes.

23. The method of claim 22 wherein said disease, condition or disorder modulated by a cannabinoid receptor antagonist is obesity, bulimia, attention deficit disorder, dementia, alcoholism, or tobacco abuse.

24. A method for treating inflammatory pain or an inflammatory disease in an animal in need thereof, said method comprising administering to said animal a therapeutically effective amount of a compound of claim 1.

25. The method of claim 24 wherein said disease is selected from the group consisting of arthritis, inflammatory bowel disease and congestive obstructive pulmonary disorder.
